(19) United States
(12) Patent Application Publication
(10) Pub. No.: US 2024/0010747 A1
(43) Pub. Date: Jan. 11, 2024

(54) ANTI-MRP4 (ENCODED BY ABCC4 GENE) ANTIBODIES AND USES THEREOF

(71) Applicant: Kenjockety Biotechnology, Inc., Tiburon, CA (US)

(72) Inventors: William Robert Arathoon, Tiburon, CA (US); Cindy Tan, San Jose, CA (US); Qianting Zhai, South San Francisco, CA (US)

(21) Appl. No.: 18/252,135

(22) PCT Filed: Oct. 29, 2021
(86) PCT No.: PCT/US2021/057309

§ 371 Date: May 8, 2023

Related U.S. Application Data
(60) Provisional application No. 63/113,682, filed on Nov. 13, 2020.

(51) Int. Cl. C07K 16/28

(52) U.S. Cl. CPC C07K 16/2896; C07K 2317/565; C07K 2317/24

(57) ABSTRACT

Provided are antibodies that target the cellular efflux pump ABCC4. Also provided are pharmaceutical compositions, nucleic acids, recombinant expression vectors, cells, and kits that include or encode such antibodies. Methods of using the antibodies for detecting presence or absence of ABCC4 expression in cells, e.g., tumor cells, level of ABCC4 expression, and/or inhibiting ABCC4 function are also disclosed. Also provided are methods for treating a subject for a cancer that include administering to the subject an anti-ABCC4 antibody disclosed herein.

### CROSS-REFERENCE TO RELATED APPLICATIONS

This application claims the benefit of priority to U.S. Provisional Patent Application No. 63/113,682 filed on Nov. 13, 2020, which application is incorporated herein by reference in its entirety.

### INCORPORATION BY REFERENCE OF SEQUENCE LISTING PROVIDED AS TEXT FILE

A Sequence Listing is provided herewith as a text file, “KNJY-007W0 SEQ LIST_ST25.K” created on Oct. 25, 2021 and having a size of 117,000 bytes. The contents of the text file are incorporated by reference herein in their entirety.

### INTRODUCTION

Drug resistance, a well-known phenomenon that results when diseases become tolerant to pharmaceutical treatments, is a major and increasing challenge in various fields of medicine, including oncology. Although many types of cancers are initially susceptible to chemotherapy, over time they can develop resistance through these and other mechanisms, including DNA mutations and metabolic changes that promote drug inhibition, degradation and enhanced efflux.

Efflux pumps (EP) are proteins expressed by living cells and have evolved to naturally expel various compounds from the cells. Members of the ATP-binding cassette (ABC) transporter family proteins are examples of EPs that enable drug efflux. Though a transporter's structure varies from protein to protein (e.g., there are 49 known members of the ABC family in humans), they are all classified by the presence of two distinct domains—a highly conserved nucleotide binding domain and a more variable transmembrane domain. Multidrug resistance protein 1 (MDR1), encoded by the ATP Binding Cassette Subfamily B Member 1 (ABCB1) gene, was the first of these to be identified and has been studied extensively. ATP Binding Cassette Subfamily C Member 4 (ABCC4) expression is increased in response to treatment with certain chemotherapeutics. Multidrug resistance protein 4 (MRP4), encoded by the ATP Binding Cassette Subfamily C Member 4 (ABCC4) gene, is involved in the cellular transport of a variety of molecules, including many anti-cancer and antiviral compounds, and has broad substrate specificity.

EPs enable tumors to develop resistance to chemotherapeutic agents. Such resistance is frequently associated with enhanced efflux of the chemotherapeutic agent from the drug resistant cells. This chemotherapy resistance is termed multi drug resistance (MDR) when it applies to more than one chemotherapeutic agent.

As such there is a need to develop reagents that may be used for assaying for expression of EPs and/or inhibiting EPs.

### SUMMARY

Provided are antibodies that bind to the cellular efflux pump multidrug resistance protein 4 (MRP4). Anti-MRP4 antibodies are referred to herein as anti-ABCC4 antibodies. Also provided are pharmaceutical compositions, nucleic acids, recombinant expression vectors, cells, and kits that include or encode such antibodies. Methods of using the antibodies for detecting presence or absence of ABCC4 expression in cells, e.g., tumor cells, level of ABCC4 expression, and/or inhibiting ABCC4 function are also disclosed. Also provided are methods for treating a subject for a cancer that include administering to the subject an anti-ABCC4 antibody disclosed herein.

### Definitions

The terms “antibody” and “immunoglobulin” include antibodies or immunoglobulins of any isotype, fragments of antibodies which retain specific binding to antigen, including, but not limited to, Fab, Fv, scFv, Fd, Fab′, Fv, F(ab′)₂, chimeric antibodies, humanized antibodies, monoclonal antibodies, single-chain antibodies, including antibodies comprising only heavy chains (e.g. VHH camelid antibodies), bispecific antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. An antibody may be monovalent or bivalent. An antibody may be conjugated to a toxic moiety, such as, a chemotherapeutic agent.

According to some embodiments, an antibody of the present disclosure is a monoclonal antibody. “Monoclonal antibody” refers to a composition comprising one or more antibodies obtained from a population of substantially homogeneous antibodies, i.e., a population the individual antibodies of which are identical except for any naturally occurring mutations that may be present in minor amounts. Monoclonal antibodies are highly specific, being directed against a single antigenic site and generally to a single epitope on an antigen. The modifier “monoclonal” indicates the character of the antibody as being obtained from a substantially homogeneous population of antibodies, and does not require that the antibody be produced by any particular method or be the only antibody in the composition.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules, including antibodies comprising only heavy chains (e.g. VHH camelid antibodies); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site comprising the three CDRs of each variable domain.

The “Fab” fragment also contains the constant domain of the light chain and the first constant domain (CH₁) of the heavy chain. Fab fragments differ from Fab′ fragments by the addition of a few residues at the carboxyl terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv”, “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, ionic, and/or hydrogen-bond interactions, including interactions such as salt bridges and water bridges. An ABCC4-specific antibody binds specifically to an epitope within a ABCC4 polypeptide. An antibody of the present disclosure specifically binds to ABCC4 on a cell surface. An antibody “specifically binds” or “preferentially binds” to a target if it binds with greater affinity, avidity, more readily, and/or with greater duration than it binds to other substances, e.g., in a sample. In certain embodiments, an antibody “specifically binds” an antigen if it binds to or associates with the antigen with an affinity or Ka (that is, an association rate constant of a particular binding interaction with units of 1/M) of, for example, greater than or equal to about 10⁴ M⁻¹. Alternatively, affinity may be defined as an equilibrium dissociation constant (KD) of a particular binding interaction with units of M. K_{D} is the equilibrium dissociation constant, a ratio of k_{off}/kₒₙ, between the antibody and its antigen. K_{D} and affinity are inversely related. In certain aspects, specific binding means the antibody binds to the antigen with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the antibody for the antigen can be readily determined using conventional techniques, e.g., by competitive ELISA (enzyme-linked immunosorbent assay), equilibrium dialysis, by using surface plasmon resonance (SPR) technology (e.g., the BIAcore 2000 or BIAcore T200 instrument, using general procedures outlined by the manufacturer); by radioimmunoassay; or the like. The epitope on the antigen may be a linear epitope formed by a contiguous stretch of amino acids or a non-linear or a conformational epitope formed by non-contiguous stretches of amino acids.

As used herein, the term “CDR” or “complementarity determining region” is intended to mean the non-contiguous antigen combining sites found within the variable region of both heavy and light chain polypeptides. CDRs are hypervariable regions and are interspersed with regions that are more conserved, termed “framework regions (FR)”. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs. A VH chain can comprise three CDRs and four FRs arranged from N-terminus to C-terminus in the following order: FR1, CDR1, FR2, CDR2, FR3, CDR3, FR4. Similarly, a VL chain can comprise three CDRs and four FRs arranged from N-terminus to C-terminus in the following order: FR1, CDR1, FR2, CDR2, FR3, CDR3, FR4.

As used herein, the term antibody encompasses a tetramer of two heavy and two light chains, wherein the heavy and light chains are interconnected by, for example, disulphide bonds. The heavy chain constant region is comprised of three domains, CH1, CH2 and CH3. The light chain constant region is comprised of one domain, CL. The variable regions of the heavy and light chains comprise binding regions that interact with antigen. The constant regions of the antibodies typically mediate the binding of the antibody to host tissues and factors, including various cells of the immune system and the first component of the complement system. The term “antibody” includes immunoglobulins of types IgA, IgG, IgE, IgD, IgM and subtypes thereof. In some embodiments, a subject antibody is an IgG isotype, e.g., IgG1.

As used herein the term “immunoglobulin” refers to a protein including one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes; and numerous immunoglobulin variable region genes. Full-length immunoglobulin light chains (about 25 kD or 214 amino acids) are encoded by a variable region gene at the N-terminus (about 110 amino acids) and a kappa or lambda constant region at the C-terminus. Full-length immunoglobulin heavy chains (about 50 kD or 446 amino acids) are encoded by a variable region gene at the N-terminus (about 116 amino acids) and one of the other aforementioned constant region genes at the C-terminus, e.g. gamma (encoding about 330 amino acids). In some embodiments, a subject antibody comprises full-length immunoglobulin heavy chain and a full-length immunoglobulin light chain.

The term “antigen-binding fragment” refers to one or more fragments of a full-length antibody that are capable of specifically binding to an antigen. Examples of binding fragments include (i) a Fab fragment (a monovalent fragment including, e.g., consisting of, the VL, VH, CL and CH1 domains; (ii) a F(ab′)₂ fragment (a bivalent fragment comprising two Fab fragments linked by a disulfide bridge at the hinge region; (iii) a Fd fragment (including, e.g., consisting of, the VH and CH1 domains); (iv) a Fv fragment (including, e.g., consisting of, the VH and VL domains of a single arm of an antibody); (v) a dAb fragment (including, e.g., consisting of, the VH domain); (vi) an isolated CDR; (vii) a single chain Fv (scFv) (including, e.g., consisting of, the VH and VL domains of a single arm of an antibody joined by a synthetic linker using recombinant means such that the VH and VL domains pair to form a monovalent molecule); (viii) diabodies (including, e.g., consisting of, two scFvs in which the VH and VL domains are joined such that they do not pair to form a monovalent molecule; the VH of each one of the scFv pairs with the VL domain of the other scFv to form a bivalent molecule).

The term “chimeric” antibody refers to an antibody in which a portion of the heavy and/or light chain is derived from a particular source or species, while the remainder of the heavy and/or light chain is derived from a different source or species.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework (FR) which represents the most commonly occurring amino acid residues in a selection of human immunoglobulin variable light chain (VL) or variable heavy chain (VH) framework sequences. Generally, the selection of human immunoglobulin VL or VH sequences is from a subgroup of variable domain sequences.

Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human CDRs and amino acid residues from human frameworks (FRs). At least a portion of a humanized antibody constant region is derived from a human antibody, e.g., a human IgG1 antibody. In preferred embodiments, the antibody molecules disclosed herein include a heavy chain comprising a variable heavy chain region as provided herein and a human IgG1 constant region having the amino acid sequence sequence set forth in UniProt: P01857-1, version 1. In preferred embodiments, the antibody molecules disclosed herein include a light chain comprising a variable light chain region as provided herein and a human light chain constant region. In preferred embodiments, the human light chain constant region is a human kappa light chain constant region having the amino acid set forth in UniProtKB/Swiss-Prot: P01834.2. In certain embodiments, the human IgG1 heavy chain constant region present in the subject antibodies may include mutations, e.g., substitutions to modulate Fc function. For example, the LALAPG effector function mutations (L234A, L235A, and P329G) or the N297A mutation may be introduced to reduce antibody dependent cellular cytotoxicity (ADCC). The numbering of the substitutions is based on the EU numbering system. The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, MD. (1991)). The “EU index as in Kabat” refers to the residue numbering of the human IgG 1 EU antibody.

A “humanized form” of an antibody, e.g., a non-human antibody, refers to an antibody that has undergone humanization.

The term “epitope” refers to a region of an antigen that is recognized by the immune system, for example by antibodies, B cells, or T cells. For example, the epitope is the specific region of the antigen to which an antibody binds.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In some embodiments, the antibody will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated antibody includes the antibody in situ within recombinant cells since at least one component of the antibody's natural environment will not be present. In some instances, isolated antibody will be prepared by at least one purification step.

The term “cytotoxic agent” as used herein refers to a substance that inhibits or prevents a cellular function and/or causes cell death or destruction. A “chemotherapeutic agent,” also referred to an “antineoplastic agent,” can be a cytotoxic agent which is used for treating a cancer or other disease or disorder.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, including in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of a target-specific antibody that, when administered to a mammal or other subject for treating a disease, is sufficient to affect such treatment for the disease. The “therapeutically effective amount” will vary depending on the antibody, the disease and its severity and the age, weight, etc., of the subject to be treated.

The term “refractory”, used herein, refers to a disease or condition that does not respond to treatment. With regard to cancer, “refractory cancer”, as used herein, refers to cancer that does not respond to treatment. A refractory cancer may be resistant at the beginning of treatment or it may become resistant during treatment. Refractory cancer may also be called resistant cancer.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples.

Percent identity between a pair of sequences may be calculated by multiplying the number of matches in the pair by 100 and dividing by the length of the aligned region, including gaps. Identity scoring only counts perfect matches and does not consider the degree of similarity of amino acids to one another. Only internal gaps are included in the length, not gaps at the sequence ends. Percent Identity=(Matches×100)/Length of aligned region (with gaps).

The phrase “conservative amino acid substitution” refers to substitution of amino acid residues within the following groups: 1) L, I, M, V, F; 2) R, K; 3) F, Y, H, W, R; 4) G, A, T, S; 5) Q, N; and 6) D, E. Conservative amino acid substitutions may preserve the activity of the protein by replacing an amino acid(s) in the protein with an amino acid with a side chain of similar acidity, basicity, charge, polarity, or size of the side chain.

Guidance for substitutions, insertions, or deletions may be based on alignments of amino acid sequences of proteins from different species or from a consensus sequence based on a plurality of proteins having the same or similar function.

The term “vector” means any molecule or entity (e.g., nucleic acid, plasmid, bacteriophage or virus) used to transfer protein coding information into a host cell.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct may include, but is not limited to, sequences that affect or control transcription, translation, and, if introns are present, affect RNA splicing of a coding region operably linked thereto.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one embodiment, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing cytoplasmic signaling sequence that is of particular use in the invention includes, but is not limited to, those derived from CD3 zeta, common FcR gamma (FCER1G), Fc gamma RIla, FcR beta (Fc Epsilon R1b), CD3 gamma, CD3 delta, CD3 epsilon, CD79a, CD79b, DAP10, and DAP12.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137).

The term “autologous” refers to any material derived from the same individual to whom it is later to be re-introduced into the individual.

An “intracellular signaling domain,” as the term is used herein, refers to an intracellular portion of a molecule. The intracellular signaling domain generates a signal that promotes an immune effector function of the CAR containing cell, e.g., a CAR-T cell. Examples of immune effector function, e.g., in a CAR-T cell, include cytolytic activity and helper activity, including the secretion of cytokines.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

### DETAILED DESCRIPTION

Provided are antibodies that bind to the cellular efflux pump ABCC4. Also provided are pharmaceutical compositions, nucleic acids, recombinant expression vectors, cells, and kits that include or encode such antibodies. Methods of using the antibodies for detecting presence or absence of ABCC4 expression in cells, e.g., tumor cells, level of ABCC4 expression, and/or inhibiting ABCC4 function are also disclosed. Also provided are methods for treating a subject for a cancer that include administering to the subject an anti-ABCC4 antibody as disclosed herein.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the methods and compositions have or will be described for the sake of grammatical fluidity with functional explanations, it is to be expressly understood that the claims, unless expressly formulated under 35 U.S.C. § 112(f), are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112(f) are to be accorded full statutory equivalents under 35 U.S.C. § 112(f).

Antibodies

As summarized above, the present disclosure provides antibodies that bind a cellular efflux pump ABCC4 expressed on surface of a mammalian cell, e.g., a human cell. ABCC4, also known as or Multidrug Resistance-Associated Protein 4 (MRP4), is an energy-dependent pump that effluxes many anti-cancer and anti-viral compounds across the plasma membrane. Human ABBC4 is a 1325-amino acid protein that is structurally similar to other ABC transporter family members, having two membrane-spanning domains, each with six transmembrane helices. While MRP4 is highly conserved across many species, it does not share significant sequence similarity with other ABC transporters. ABCC4 is involved in transporting nucleoside/nucleotide analogs and topoisomerase inhibitors. Overexpression of ABCC4 confers resistance to nucleoside analogs cytarabine and troxacitabine.

In some embodiments, the antibodies disclosed herein bind to one or more sites on an extracellular region of ABCC4. In certain embodiments, the anti-ABCC4 antibodies of the present disclosure bind to human ABCC4. In certain embodiments, the anti-ABCC4 antibodies of the present disclosure bind to human ABCC4 expressed on the cell surface of a human cell, e.g., cancer cell.

In addition, antibodies of the present disclosure may have one or more of the following properties:
i) Inhibits efflux from ABCC4;
ii) increases sensitivity of cancer cell to treatment with a chemotherapeutic agent thereby lowering the IC50 of the chemotherapeutic agent by at least a factor of 2;
iii) binds to human and cynomolgus ABCC4 on cell surface;
iv) is effective in in vitro cell killing assays;
v) is effective in inhibiting tumor growth even in absence of chemotherapy; and
vi) has an affinity for ABCC4 in a lower range such that it binds to cancer cells that express ABCC4 at a higher level as compared to non-cancer cells and binds significantly less to non-cancer cells.

As used herein, EC50 refers to the concentration of an antibody that provides half maximal response (e.g., half of the maximum fluorescence intensity). The antibodies of the present disclosure may have an EC50 of 100 nM or lower, e.g., 100 nM-4 nM, 80 nM-4 nM, 60 nM-4 nM, 40 nM-4 nM, 30 nM-4 nM, 20 nM-4 nM, 15 nM-4 nM, or 10 nM-4 nM. EC50 of a test antibody many be determined by flow cytometry or ELISA. For example, flow cytometry may involve contacting a cell expressing ABCC4 (e.g. human ABCC4) with the antibody in a flow cytometry buffer, where the antibody is serially diluted, and incubating at room temperature or 4° C. for a period of time sufficient for the antibody to bind to the cells (e.g. 10 min-1 hr). After incubating, the cells may optionally be washed to remove and non-specifically bound antibody and/or the cells may be contacted with a fluorescently labeled secondary antibody that specifically binds to the test antibody. After incubation, the fluorescently labeled secondary antibody may be removed and the cells washed. The washed cells may be sorted by flow cytometry and the number of cells bound to the fluorescently labeled secondary antibody counted. The concentration that provides half maximal response (e.g., half of the maximum fluorescence intensity) is measured as the EC50. In variations of the flow cytometry assay, the cell may be a 293T cell or another cancer cell line overexpressing ABCC4.

The IC50 of a test antibody may be determined by measuring inhibition of cell growth. IC50 may be measured by using the test antibody alone to determine the concentration of the antibody that produced half maximal response. The IC50 of a chemotherapeutic agent may be measured in the absence and in the presence of the test antibody to determine the effect of the antibody on the IC50 chemotherapeutic agent. The chemotherapeutic agent may be cytarabine, troxacitabine, doxorubicin, daunorubicin, etoposide, vincristine, etc. The cell may be a cancer cell line. Cells may be contacted with antibody alone if determining the IC50 of the antibody, wherein the antibody is tested at serial dilutions. Cells may be contacted with antibody and the chemotherapeutic agent to determine the effect of the antibody on the IC50 of the agent, where the agent is tested at serial dilutions. The cells may be incubated at 37° C. for a period of time (e.g. 24 hr-84 hr) and cell viability assessed using standard reagents and methods. The antibodies disclosed herein may increase sensitivity of cancer cell to treatment with a chemotherapeutic agent thereby lowering the IC50 of the chemotherapeutic agent by at least a factor of 2. In certain embodiments, the antibodies of the present disclosure may lower the IC50 of the chemotherapeutic agent by factor of 2 or more, e.g., factor of 3 or more, factor of 4 or more, factor of 5 or more, factor of 6 or more, factor of 6 or more, factor of 7 or more, factor of 8 or more, factor of 9 or more, or factor of 10 or more, e.g., by a factor of 2 to 10.

In certain embodiments, one or more of the anti-ABCC4 antibodies disclosed herein bind to both human and cynomolgus ABCC4. This property may be utilized in determining safety and/or dose of the antibody using an animal model.

In certain embodiments, the anti-ABCC4 antibodies disclosed herein are specific for ABCC4 and do not show significant binding to other antigens, e.g., other efflux pumps, such as, ABCB1, ABCG2, ABCC1, etc.

In some embodiments, one or more of the subject antibodies may, when bound to a cell expressing ABCC4, prevent the functioning of the cellular ABCC4 protein. Accordingly, one or more antibodies of the present disclosure may inhibit efflux by the ABCC4 protein, including e.g., where efflux is reduced by 5% or more, including e.g., 10% or more, 15% or more, 20% or more, 25% or more, 30% or more, 40% or more, 50% or more, 60% or more, 70% or more, 80% or more, or 90% or more, as compared to efflux by ABCC4 in the absence of the subject antibody. In some embodiments, the subject antibodies may, when bound to a cell expressing ABCC4 may otherwise impede the action of ABCC4 by other mechanisms, e.g., rendering ABCC4 leaky which in turn may enhance uptake of a chemotherapeutic agent and/or decrease viability of the cell.

In certain embodiments, an anti-ABCC4 antibody that competes for binding to ABCC4 with an antibody comprising heavy chain complementarity determining regions (HCDRs) and light chain CDRs (LCDRs) of the variable heavy chain (VH) region and the variable light chain (VL) region pair, respectively, of an antibody listed in Table 2 is provided. For example, in one embodiment, an anti-ABCC4 antibody of the present disclosure competes for binding to ABCC4 with the C4.844 antibody listed in Table 2. In certain embodiments, HCDRs 1-3 and LCDRs 1-3 are defined as per Kabat nomenclature.

In certain embodiments, the anti-ABCC4 antibody comprises the HCDR1, HCDR2, and HCDR3 of the VH region of the antibody listed in Table 2. In certain embodiments, the HCDR1, HCDR2, and HCDR3 are defined as per Kabat nomenclature. For example, in one embodiment, the anti-ABCC4 antibody of the present disclosure that competes for binding to ABCC4 with the C4.844 antibody listed in Table 2 comprises the HCDR1, HCDR2, and HCDR3 of the VH region of the C4.844 antibody.

Any suitable approach for determining whether a first antibody competes with a second antibody for binding to ABCC4 may be employed. Whether a first antibody “competes with” a second antibody for binding to an antigen may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is added. One of the two antibodies is labelled. If the labeled antibody and the unlabeled antibody bind to separate and discrete sites on the antigen, the labeled antibody will bind to the same level whether or not the suspected competing antibody is present. However, if the sites of interaction are identical or overlapping, the unlabeled antibody will compete, and the amount of labeled antibody bound to the antigen will be lowered. If the unlabeled antibody is present in excess, very little, if any, labeled antibody will bind.

For purposes of the present disclosure, competing antibodies are those that decrease the binding of an antibody to the antigen by about 30% or more, about 40% or more, about 50% or more, about 60% or more, about 70% or more, about 80% or more, about 85% or more, about 90% or more, about 95% or more, or about 99% or more. Details of procedures for carrying out such competition assays are well known in the art and can be found, for example, in Harlow and Lane, Antibodies, A Laboratory Manual, Cold Spring Harbor Laboratory Press, Cold Spring Harbor, New York, 1988, 567-569, 1988, ISBN 0-87969-314-2. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the antigen may be titrated. The results may be plotted, and the concentrations necessary to achieve the desired degree of binding inhibition may be compared.

In certain embodiments, an antibody that specifically binds to ABCC4 comprises (i) HCDRs 1-3 and light chain CDRs (LCDRs 1-3) of a pair of variable heavy chain (VH) region and variable light chain (VL) region of an antibody listed in Table 2; (ii) HCDRs 1-3 of a VH region of an antibody listed in Table 2; (iii) LCDRs 1-3 of a VH region of an antibody listed in Table 2; or (iv) HCDRs 1-3 of a VH region of a first antibody listed in Table 2 and LCDRs 1-3 of a VL region of second antibody listed in Table 2. The HCDRs and the LCDRs may be defined based on the Kabat nomenclature.

In certain embodiments, an antibody of the present disclosure that binds specifically to human ABCC4 comprises the HCDR1, HCDR2, and HCDR3 sequences and the LCDR1, LCDR2, and LCDR3 sequences of an antibody listed in Table 2. In addition to binding to human ABCC4, one or more of the antibodies provided herein may bind to ABCC4 from other mammalian species, such as, mouse, monkey, chimpanzee, etc. The antibodies may be raised in mouse or rat. The antibodies listed in Table 2 were raised in rats.

TABLE 2
From left to right, 1^{st} column: Anti-ABCC4 antibody name, 2^{nd} column: VH region,
3^{rd} column: HCDR1, 4^{th} column: HCDR2, 5^{th} column: HCDR3, 6^{th} column: VL region, 7^{th} column:
LCDR1, 8^{th} column: LCDR2, 9^{th} column: LCDR3.

| | Heavy Chain Variable | | | | | | | |
|---|---|---|---|---|---|---|---|---|
| Antibody | Region | CDRH1 | CDRH2 | CDRH3 | Light Chain Variable Region | CDRL1 | CDRL2 | CDRL3 |
| C4.884 | EVQLVESGPGLVQPSQTLSLT | SNSVH | AIWSGGSTD | LIFPY (SEQ | DVVMTQTPSILSGTLGRSVSISC | RSSQSLLD | LVSNLGS | MQATHVPF |
| | CTVSGFSLTSNSVHWVRQPP | (SEQ ID | YNSALKS | ID NO: 92) | RSSQSLLDSDGTTYLYWFLQRP | SDGTTYLY | (SEQ ID | T (SEQ ID |
| | GKGLEWIGAIWSGGSTDYNS | NO: 37) | (SEQ ID | | GQSPQRLIYLVSNLGSGVPNRFS | (SEQ ID | NO: 182) | NO: 205) |
| | ALKSRLSISRDTSKSQVFLKM | | NO: 61) | | GSGSGTDFTLKISGVEAEDLGVY | NO: 155) | | |
| | NSLQTEDTAIYFCTGLIFPYW | | | | YCMQATHVPFTFGSGTKLEIK | | | |
| | GQGASVTVSS (SEQ ID | | | | (SEQ ID NO: 124) | | | |
| | NO: 1) | | | | | | | |
| C4.886 | EVKLLESGGDLLQPGRSLKLSC | DYNMA | TILYDGSRTY | EFGYGSSH | DVVLTQTPVSLPVSLGGQASISC | RSSQSLVH | RISNRFS | FQSTHFPHT |
| | AASGFTFNDYNMAWVRQAP | (SEQ ID | YRDSVKG | WFAC (SEQ | RSSQSLVHSNGNTYLHWYLQK | SNGNTYL | (SEQ ID | (SEQ ID |
| | KKGLQWVATILYDGSRTYYR | NO: 38) | (SEQ ID | ID NO: 93) | PGQSPQLLISRISNRFSGVPDRF | H (SEQ ID | NO: 183) | NO: 206) |
| | DSVKGRFTISRDNSKSTLYLQ | | NO: 62) | | SGSGSGTDFTLKISRVEPEDLGD | NO: 156) | | |
| | MDSLRSEDTATYYCTTEFGYG | | | | YYCFQSTHFPHTFGAGTKLEIK | | | |
| | SSHWFACWGQGTLVTVSS | | | | (SEQ ID NO: 125) | | | |
| | (SEQ ID NO: 2) | | | | | | | |
| C4.925 | EVQLVESGGGLVQPGRSLKLS | DYYMA | YINYDGGST | ENYGYNSF | DIQVTQSPASLSASLGETISIECLT | LTSEDISSY | GANSLQ | QQSYKFPLT |
| | CAASGFTFSDYYMAWVRQA | (SEQ ID | HYGDSVKG | DY (SEQ ID | SEDISSYLTWYQQKPGKSPHLLI | LT (SEQ ID | A (SEQ ID | (SEQ ID |
| | PTKGLEWVAYINYDGGSTHY | NO: 39) | (SEQ ID | NO: 94) | YGANSLQAGVPSRFSGSGSGTQ | NO: 157) | NO: 184) | NO: 207) |
| | GDSVKGRFTISRDNEKSTLYL | | NO: 63) | | YSLKISSMQPEDEGDYFCQQSY | | | |
| | QMNSLRSEDMATYYCAREN | | | | KFPLTFGSGTKLELK (SEQ ID | | | |
| | YGYNSFDYWGQGASVTVSS | | | | NO: 126) | | | |
| | (SEQ ID NO: 3) | | | | | | | |
| C4.927 | EVQLVESGPGLVQPSRTLSLT | SYGVS | AIWSGGSTD | GATEGIDY | AIQVTQSPTSLSASLGDRVTLTC | RASQDIN | YASTLQS | LQGYSLYT |
| | CTVSGFSLTSYGVSWVRQPP | (SEQ ID | YNSALKS | (SEQ ID | RASQDINNKMAWYQQKPGEV | NKMA | (SEQ ID | (SEQ ID |
| | GKGLEWIAAIWSGGSTDYNS | NO: 40) | (SEQ ID | NO: 95) | PQLLIYYASTLQSGTPSRFSGSGS | (SEQ ID | NO: 185) | NO: 208) |
| | ALKSRLSISRDTSKSQVLLKM | | NO: 61) | | GTDFSFTISHLQSEDFATYYCLQ | NO: 158) | | |
| | NSLQTEDTAMYFCARGATEG | | | | GYSLYTFGAGTKLELK (SEQ ID | | | |
| | IDYWGQGASVTVSS (SEQ ID | | | | NO: 127) | | | |
| | NO: 4) | | | | | | | |
| C4.947 | EVKLVESGGGLVQPGGSMRL | DFYMN | FIRNKANAYT | SNYGFDY | DVAMTQTPPSLSVAIGQSVSISC | KSSQSLVY | QVSNLDS | AQTTHFPFT |
| | SCAASGFTFTDFYMNWIRQP | (SEQ ID | TAYNPSVKG | (SEQ ID | KSSQSLVYSDGKTYLHWLLQSS | SDGKTYLH | (SEQ ID | (SEQ ID |
| | AGKAPEWLGFIRNKANAYTT | NO: 41) | (SEQ ID | NO: 96) | GRSPKRLIYQVSNLDSGVPDRFS | (SEQ ID | NO: 186) | NO: 209) |
| | AYNPSVKGRFTISRDNAQNM | | NO: 64) | | GTGSQKDFTLKISRVEAKDLGVY | NO: 159) | | |
| | LYLQMNTLRAEDTATYYCARS | | | | YCAQTTHFPFTFGSGTKLEIK | | | |
| | NYGFDYWGQGVMVTVSS | | | | (SEQ ID NO: 128) | | | |
| | (SEQ ID NO: 5) | | | | | | | |
| C4.953 | QVTLKESGPGILQPSQTLSLTC | TYGMGVG | NIWWDDDK | IGSGNSEFD | DVVLTQSPSSLSASLGDRVTITC | RASQDIG | GATNLAA | LQSIQYPFT |
| | TFSGFSLSTYGMGVGWIRQP | (SEQ ID | SYNPSLKN | Y (SEQ ID | RASQDIGNYLTWFQQKPGKSP | NYLT (SEQ | (SEQ ID | (SEQ ID |
| | SGKALEWLANIWWDDDKSY | NO: 42) | (SEQ ID | NO: 97) | RRMIYGATNLAAGVPSRFSGSR | ID | NO: 187) | NO: 210) |
| | NPSLKNRLTISKGTSNNQAFL | | NO: 65) | | SGSDYSLTISSLESEDVADYHCL | NO: 160) | | |
| | KITSVDTADSATYHCARIGSG | | | | QSIQYPFTFGSGTKLEIK (SEQ | | | |
| | NSEFDYWGQGVMVTVSS | | | | ID NO: 129) | | | |
| | (SEQ ID NO: 6) | | | | | | | |
| C4.956 | QLKESGPGLVKPSQSLSLTCSV | YISYSGFTN | YISYSGFTNY | TRGYNPFPY | AIQVTQSPTSLSASLGDRVTLTC | RASQDIN | YASTLQS | LQGYSLYT |
| | TGYTITSGYDWTWLRKFPGN | YNPSLRS | NPSLRS | (SEQ ID | RASQDINNKMAWYQQKPGEV | NKMA | (SEQ ID | (SEQ ID |
| | KMEWMGYISYSGFTNYNPSL | (SEQ ID | (SEQ ID | NO: 98) | PQLLIYYASTLQSGTPSRFSGSGS | (SEQ ID | NO: 185) | NO: 208) |
| | RSRISITRDTSKNQFFLQLNSV | NO: 43) | NO: 43) | | GTDFSFTISHLQSEDFATYYCLQ | NO: 158) | | |
| | TAEDTATYFCTRTRGYNPFPY | | | | GYSLYTFGAGTKLELK (SEQ ID | | | |
| | WGQGTLVTVSS (SEQ ID | | | | NO: 127) | | | |
| | NO: 7) | | | | | | | |
| C4.957 | EVQLKESGPGLVQPSQTLSLT | NYGVS | AIWSGGSTG | SGVEGPPF | AIQVTQSPTSLSASLGDRVTLTC | RASQDIN | YASTLQS | LQGYSLYT |
| | CTVSGFSLTNYGVSWVRQPP | (SEQ ID | YNSALKS | DY (SEQ ID | RASQDINNKMAWYQQKPGEV | NKMA | (SEQ ID | (SEQ ID |
| | GKGLEWIGAIWSGGSTGYNS | NO: 44) | (SEQ ID | NO: 99) | PQLLIYYASTLQSGTPSRFSGSGS | (SEQ ID | NO: 185) | NO: 208) |
| | ALKSRLSISRDTSKSQVLLKM | | NO: 66) | | GTDFSFTISHLQSEDFATYYCLQ | NO: 158) | | |
| | NSLQTEDTAMYFCARSGVEG | | | | GYSLYTFGAGTKLELK (SEQ ID | | | |
| | PPFDYWGQGASVTVSS (SEQ | | | | NO: 127) | | | |
| | ID NO: 8) | | | | | | | |
| C4.961 | EVQLKESGPGLVQPSQTLSLT | NYHVH | VMWSDGDT | GGVYFDY | DIVMTQSPSSLAVSAGETVTINC | KSSQSLLYS | WASTRQ | QHYFDTPY |
| | CAVSGFSLTNYHVHWVRQPP | (SEQ ID | SYNSALKS | (SEQ ID | KSSQSLLYSGNQKNSLAWYQQ | GNQKNSL | S (SEQ ID | T (SEQ ID |
| | GKGLEWMGVMWSDGDTSY | NO: 45) | (SEQ ID | NO: 100) | KPGQSPKLLIYWASTRQSGVPD | A (SEQ ID | NO: 188) | NO: 211) |
| | NSALKSRLSISRDTSKSQFFLK | | NO: 67) | | RFIGSGSGTDFTLTFSSVQAEDL | NO: 161) | | |
| | MNSLQTEDTATYYCARGGVY | | | | AIYYCQHYFDTPYTFGAGTKLEL | | | |
| | FDYWGQGASVTVSS (SEQ ID | | | | K (SEQ ID NO: 130) | | | |
| | NO: 9) | | | | | | | |
| C4.970 | EVQLVESGPGLVQPSQTLSLT | SYNVH | VIWNTGDTR | VIWNTGDT | DIKMIQSPSSLSASLGERVTISCR | RASENINN | HTSRLQP | QQGYTPPT |
| | CTVAGFSLTSYNVHWVRQPP | (SEQ ID | YDSALKS | RYDSALKS | ASENINNYLSWYQKKEDGSVKL | YLS (SEQ | (SEQ ID | (SEQ ID |
| | GKGLEWMGVIWNTGDTRY | NO: 46) | (SEQ ID | (SEQ ID | LIYHTSRLQPGAPSRFSGSGSGK | ID | NO: 189) | NO: 212) |
| | DSALKSRLSISKDTSKSQVFLN | | NO: 68) | NO: 68) | DYSLTISGLDSEDIATYYCQQGY | NO: 162) | | |
| | MNSLQTEDTATYYCARDDNV | | | | TPPTFGAGTKLELK (SEQ ID | | | |
| | YYGMHAMDAWGQGASVTV | | | | NO: 131) | | | |
| | SS (SEQ ID NO: 10) | | | | | | | |
| C4.972 | EVQLKESGPGLVQPSETLSLTC | SYNVH | VMWSGGST | DRGGYYYD | EIVIIQSPALAVSLGQRATISCKT | KTNQNVD | LASNLAS | QQSRNLPF |
| | TVSGFSLTSYNVHWVRQPPG | (SEQ ID | DYSSALKS | GYPHDFVY | NQNVDYYGNSYIHWYQQKPG | YYGNSYIH | (SEQ ID | T (SEQ ID |
| | KGLEWMGVMWSGGSTDYS | NO: 46) | (SEQ ID | (SEQ ID | QQPKLLIYLASNLASGIPARFSG | (SEQ ID | NO: 190) | NO: 213) |
| | SALKSRLSISRDTSKNQVFLK | | NO: 69) | NO: 101) | RGSGTDFTLTIDPVEADDPATYY | NO: 163) | | |
| | MNSLQSEDTTTYYCARDRGG | | | | CQQSRNLPFTFGSGTKLELK | | | |
| | YYYDGYPHDFVYWGQGVM | | | | (SEQ ID NO: 132) | | | |
| | VTVSS (SEQ ID NO: 11) | | | | | | | |
| C4.976 | EVKLLESGGGLVQPGRSLKVS | DYYMA | YINYDGGSTY | RDYGSKFD | EIVIIQSPALAVSLGQRATISCKT | KTNQNVD | LASNLAS | QQSRNLPF |
| | CVASGFIFSDYYMAWVRQAP | (SEQ ID | YGDSVKG | Y (SEQ ID | NQNVDYYGNSYIHWYQQKPG | YYGNSYIH | (SEQ ID | T (SEQ ID |
| | TKGLEWVAYINYDGGSTYYG | NO: 39) | (SEQ ID | NO: 102) | QQPKLLIYLASNLASGIPARFSG | (SEQ ID | NO: 190) | NO: 213) |
| | DSVKGRFTISRDNAKSSLYLQ | | NO: 70) | | RGSGTDFTLTIDPVEADDPATYY | NO: 163) | | |
| | MNSLRFEDMATYYCARRDY | | | | CQQSRNLPFTFGSGTKLELK | | | |
| | GSKFDYWGQGVMVTVSS | | | | (SEQ ID NO: 132) | | | |
| | (SEQ ID NO: 12) | | | | | | | |
| C4.981 | EVQLVESGGGLVQPKESLKIS | NAAMY | RIRTKPNNY | YNSGFLDC | DVVLTQTPSILSATIGQSVSISCR | RSSQSLLD | LVSNLGS | MQATHSYT |
| | CAASGFTFSNAAMYWVRQA | (SEQ ID | ATYYADSVK | (SEQ ID | SSQSLLDSDGNTYLYWFLQRPG | SDGNTYLY | (SEQ ID | (SEQ ID |
| | P (SEQ ID NO: 13) | NO: 47) | G (SEQ ID | NO: 103) | QSPQRLIYLVSNLGSGVPNRFSG | (SEQ ID | NO: 182) | NO: 214) |
| | | | NO: 71) | | SGSGTDFTLKISGVEAEDLGVFY | NO: 164) | | |
| | | | | | CMQATHAPFTFGSGTKLEVK | | | |
| | | | | | (SEQ ID NO: 133) | | | |
| C4.985 | EVQLVESGGGLVQPGRSLKLS | KNWMA | SITNTGGNT | ERIGSGYYV | DTVLTQSPALTVSPGERVTISCR | RASESVTS | LASDLES | QQSWIDPP |
| | CTASGFTFNKNWMAWIRQA | (SEQ ID | YYPDSVKG | MDA (SEQ | ASESVTSFMNWYQQKPGQQP | FMN (SEQ | (SEQ ID | T (SEQ ID |
| | PGKGLEWVASITNTGGNTYY | NO: 48) | (SEQ ID | ID NO: 104) | KLLIYLASDLESGVPARFSGSGSG | ID | NO: 191) | NO: 215) |
| | PDSVKGRFTVSRDQTISTLYL | | NO: 72) | | ADFTLTIDPVEADDTATYYCQQ | NO: 165) | | |
| | QMNSLRSEDTATYFCTRERIG | | | | SWIDPPTFGTGTKLELK (SEQ ID | | | |
| | SGYYVMDAWGRGASVTVSS | | | | NO: 134) | | | |
| | (SEQ ID NO: 14) | | | | | | | |
| C4.987 | EVQLVESGGGLVQPGRSMKL | NYYMA | SISTGGGNT | GEDRYKGV | DVQMTQSPSNLAASPGESVSIN | KASKSISKY | SGSTLQS | QHHNEYPP |
| | SCAASGFTFSNYYMAWVRQ | (SEQ ID | HYRDSVKG | FAY (SEQ | CKASKSISKYLAWYQQKPGKAN | LA (SEQ ID | (SEQ ID | T (SEQ ID |
| | APTKGLEWVASISTGGGNTH | NO: 49) | (SEQ ID | ID NO: 105) | KLLIYSGSTLQSGTPSRFSGSGSG | NO: 166) | NO: 192) | NO: 216) |
| | YRDSVKGRFTISRDNAKSTLYL | | NO: 73) | | TDFTLTIRNLEPEDFGLYYCQHH | | | |
| | QMDSLRSEETATYYCARGED | | | | NEYPPTFGAGTKLELK (SEQ ID | | | |
| | RYKGVFAYWGQGTLVTVSS | | | | NO: 135) | | | |
| | (SEQ ID NO: 15) | | | | | | | |
| C4.991 | EVKLVESGGGLVQPGRSLQV | DYYMA | YINYDGGSTY | RDYGSKFD | DIVMTQSPSLLSASVGDRVTLTC | KGSQNIN | KTNSLHT | YQYNNGYT |
| | SCVASGFIFTDYYMAWVRQA | (SEQ ID | YGDSVKG | N (SEQ ID | KGSQNINNFLAWYQQKRGEAP | NFLA (SEQ | (SEQ ID | (SEQ ID |
| | PTKGLEWVAYINYDGGSTYY | NO: 39) | (SEQ ID | NO: 106) | KLLIYKTNSLHTGIPSRFSGSGSG | ID | NO: 193) | NO: 217) |
| | GDSVKGRFTISRDNAKSTLYL | | NO: 70) | | TEYTLTISSLHSEDLATYYCYQYN | NO: 167) | | |
| | QMNSLRSEDMATYYCARRD | | | | NGYTFGAGTKLELK (SEQ ID | | | |
| | YGSKFDNWGQGTLVTVSS | | | | NO: 136) | | | |
| | (SEQ ID NO: 16) | | | | | | | |
| C4.994 | QIQLVQSGPELKKPGESVKISC | DYAIH | WISTYTGKTT | GGLQWFD | EILLTQSPTAMAASPGEKVTITC | RASFRVSY | DTSKLAS | QQWTSNS |
| | KASGYTFTDYAIHWVTQAPG | (SEQ ID | YTDDFKG | Y (SEQ ID | RASFRVSYMHWYQQKPGASPK | MH (SEQ | (SEQ ID | RT (SEQ ID |
| | KGLKWMGWISTYTGKTTYTD | NO: 50) | (SEQ ID | NO: 107) | PWIYDTSKLASGVPTRFSGSGS | ID | NO: 194) | NO: 218) |
| | DFKGRFVFSLDASASTANLQI | | NO: 74) | | GTSYSFTISSMETEDTATYYCQQ | NO: 168) | | |
| | NNLKNEDTAAYFCARGGLQ | | | | WTSNSRTFGGGTKLVLK (SEQ | | | |
| | WFDYWGQGTLVTVSS (SEQ | | | | ID NO: 137) | | | |
| | ID NO: 17) | | | | | | | |
| C4.1000 | QVQLTESGPGLVQPSQTLSLT | SYGVS | AIWSGGGTN | GDWFAY | DIQMTQSPPSLSASLGDKVTITC | QASQNIN | YTSTLES | LQYVNLYT |
| | CTISGFSLTSYGVSWVRQPPG | (SEQ ID | YDSALKS | (SEQ ID | QASQNINKYIAWFQQKPGKAP | KYIA (SEQ | (SEQ ID | (SEQ ID |
| | KGLEWIGAIWSGGGTNYDSA | NO: 40) | (SEQ ID | NO: 108) | RLLIRYTSTLESGTPSRFSGSGSG | ID | NO: 195) | NO: 219) |
| | LKSRLSISRDTSKSQVLLKMNS | | NO: 75) | | RDYSFSISTVESEDIASYYCLQYV | NO: 169) | | |
| | LQTEDTAMYFCASGDWFAY | | | | NLYTFGAGTKLELK (SEQ ID | | | |
| | WGQGTLVTVSS (SEQ ID | | | | NO: 138) | | | |
| | NO: 18) | | | | | | | |
| C4.1006 | EVQLTESGPGLVQPSQTLSLT | SYDVH | RMQNGGST | SFIIRGY | DIVMTQGALPNPVPSGESASIT | QSSKSLLYS | WMSIRA | QQFLEFPYT |
| | CTVSGFSLTSYDVHWVRQPP | (SEQ ID | DYNSALKS | (SEQ ID | CQSSKSLLYSDGKTYLNWYLQR | DGKTYLN | S (SEQ ID | (SEQ ID |
| | GKGLEWMGRMQNGGSTDY | NO: 51) | (SEQ ID | NO: 109) | PGQSPQLLIYWMSIRASGVSDR | (SEQ ID | NO: 196) | NO: 220) |
| | NSALKSRLSISRDTSKSQVFLK | | NO: 76) | | FSGSGSGTDFTLKISRVEAEDVG | NO: 170) | | |
| | MNSVQTEDTAMYFCVRSFIIR | | | | VYYCQQFLEFPYTFGAGTKLEIK | | | |
| | GYWGQGASVTVSS (SEQ ID | | | | (SEQ ID NO: 139) | | | |
| | NO: 19) | | | | | | | |
| C4.1047A | EVQLMESGPGLVQPSQTLSLT | SNNIN | AISGGGSAFY | AISGGGSAF | ETVMTQTPSSLAVSAGETVTIN | RSSQSLLYS | WSSTRQ | QQYYDTPL |
| | CTVSGLSLISNNINWVRQPPG | (SEQ ID | NSALKS | YNSALKS | CRSSQSLLYSGNQKNYLAWYQ | GNQKNYL | Y (SEQ ID | T (SEQ ID |
| | KGLEWIGAISGGGSAFYNSAL | NO: 52) | (SEQ ID | (SEQ ID | QKPGQSPKLLIYWSSTRQYGVP | A (SEQ ID | NO: 197) | NO: 221) |
| | KSRLSFSRDTSKSQVFLKMNS | | NO: 77) | NO: 77) | DRFIGSGSGTDFTLTISSVQAED | NO: 171) | | |
| | LQTEDTAIYFCARGGSEDPDY | | | | LAIYYCQQYYDTPLTFGSGTKLE | | | |
| | FDYWGQGVMVTVSS (SEQ | | | | LK (SEQ ID NO: 140) | | | |
| | ID NO: 20) | | | | | | | |
| C4.1052 | EVQLMESGPGLVQPSQTLSLT | SNNIN | AISGGGSAFY | GGSEDPDY | DIVMTQSPSSLAVSAGETVTINC | KSSQSLLYS | WSSTRQS | QQYYDTPL |
| | CTVSGLSLISNNINWVRQPPG | (SEQ ID | NSALKS | FDY (SEQ | KSSQSLLYSGNQKNYLAWYQQ | GNQKNYL | (SEQ ID | T (SEQ ID |
| | KGLEWIGAISGGGSAFYNSAL | NO: 52) | (SEQ ID | ID NO: 110) | KPGQSPKLLIYWSSTRQSGVPD | A (SEQ ID | NO: 198) | NO: 221) |
| | KSRLSFSRDTSKSQVFLKMNS | | NO: 77) | | RFMGSGSGTDFTLTISSVQAED | NO: 172) | | |
| | LQTEDTAIYFCARGGSEDPDY | | | | LAIYYCQQYYDTPLTFGSGTKLEI | | | |
| | FDYWGQGVMVTVSS (SEQ | | | | K (SEQ ID NO: 141) | | | |
| | ID NO: 20) | | | | | | | |
| C4.1054 | QVQLLQSGAALVKPGASVKM | DYWVS | EIYPNNGAT | RKYGYNW | DIVLTQSPALAVSLGQRATISCR | RARQSVSI | RASNLAS | QQSRDSP |
| | SCKPSGYTFTDYWVSWVKQS | (SEQ ID | NFNEKFKG | NFDY (SEQ | ARQSVSISRYNLMHWYQQKPG | SRYNLMH | (SEQ ID | WT (SEQ ID |
| | HGKSLEWIGEIYPNNGATNF | NO: 53) | (SEQ ID | ID NO: 111) | QQPKVLIYRASNLASGIPARFSG | (SEQ ID | NO: 199) | NO: 222) |
| | NEKFKGKATLTVDKSTSTAYM | | NO: 78) | | SGSGTDFTLTIDPVQADDIATYY | NO: 173) | | |
| | ELSRLTSEDSAIYYCPSRKYGY | | | | CQQSRDSPWTFGGGTKLELK | | | |
| | NWNFDYWGQGTMVTVSS | | | | (SEQ ID NO: 142) | | | |
| | (SEQ ID NO: 21) | | | | | | | |
| C4.1059 | QVQLLQSGAALVKPGASVKM | DYWVS | EIYPNNGAT | RKYGYNW | DIVLTQSPALAVSLGQRATISCW | WASQSVSI | RASNLAS | QQSGESPW |
| | SCKPSGYTFTDYWVSWVKQS | (SEQ ID | NFNEKFKG | NFDY (SEQ | ASQSVSISRYNLMHWYQQKPG | SRYNLMH | (SEQ ID | T (SEQ ID |
| | HGKSLEWIGEIYPNNGATNF | NO: 53) | (SEQ ID | ID NO: 111) | QQPKLLIYRASNLASGIPARFSG | (SEQ ID | NO: 199) | NO: 223) |
| | NEKFKGKATLTVDKSTSTAYM | | NO: 78) | | SGSGTDFTLTINPVQADDIATYY | NO: 174) | | |
| | ELSRLTSEDSAIYYCPSRKYGY | | | | CQQSGESPWTFGGGTKLELK | | | |
| | NWNFDYWGQGTMVTVSS | | | | (SEQ ID NO: 143) | | | |
| | (SEQ ID NO: 21) | | | | | | | |
| C4.1069 | GLVQPSQTLSLTCTVSGFSLN | SNGVS | EISSGGSAFY | GGTEDPDY | DIVLTQSPSSLAVSAGETVTINCK | KSSQSLLYS | WASTRQ | QQYYDTPV |
| | SNGVSWVRQPPGKGLEWIV | (SEQ ID | NSALKS | FDY (SEQ | SSQSLLYSGNQKNYLAWYQQT | GNQKNYL | S (SEQ ID | T (SEQ ID |
| | EISSGGSAFYNSALKSRLSISR | NO: 54) | (SEQ ID | ID NO: 112) | PGQSPKLLIYWASTRQSGVPDR | A (SEQ ID | NO: 188) | NO: 224) |
| | DTSKSQVFLKMNSLQTEDTAI | | NO: 79) | | FIGSGSGTDFILTITSVQAEDLAIY | NO: 172) | | |
| | YFCTRGGTEDPDYFDYWGQ | | | | YCQQYYDTPVTFGSGTKLELK | | | |
| | GASVTVSS (SEQ ID NO: 22) | | | | (SEQ ID NO: 144) | | | |
| C4.1071 | EVQWKESGPGLVQPSQTLSL | SYGVS | AIWSGGGTD | NHYFDY | AIQVTQSPNSLSASLGDRVTLTC | RASQDIN | SASTLQS | LQGYSLYT |
| | TCTVSGFSLTSYGVSWVRQP | (SEQ ID | YNSALKS | (SEQ ID | RASQDINNKMAWYQQKPGEV | NKMA | (SEQ ID | (SEQ ID |
| | PGKGLEWIGAIWSGGGTDY | NO: 40) | (SEQ ID | NO: 113) | PQLLIYSASTLQSGTPSRFSGSGS | (SEQ ID | NO: 200) | NO: 208) |
| | NSALKSRLSISRDTSKSQVLLK | | NO: 80) | | GTDFSFTISHLQSEDFATYYCLQ | NO: 158) | | |
| | MNSLQTEDTAMYFCAGNHY | | | | GYSLYTFGAGTKLELKRADAAP | | | |
| | FDYWGQGTMVTVSS (SEQ | | | | (SEQ ID NO: 145) | | | |
| | ID NO: 23) | | | | | | | |
| C4.1073 | EVQMKESGPGLVQPSQTLSL | SYHVH | VMWSDGDT | DRDVLYYD | DIQMTQSPSNLAASPGESVSIN | KASKSISKF | SASTLQS | QQHNEYPL |
| | TCTVSGFSLTSYHVHWVRQP | (SEQ ID | SYNSALKS | GDFSPFVM | CKASKSISKFLAWYQQKSGKAN | LA (SEQ ID | (SEQ ID | T (SEQ ID |
| | PGKGLEWMGVMWSDGDTS | NO: 55) | (SEQ ID | DA (SEQ ID | KLLIYSASTLQSGTPSRFSGSGSG | NO: 175) | NO: 200) | NO: 225) |
| | YNSALKSRLSINRDTSQSRVFL | | NO: 67) | NO: 114) | TDFTLTIRNLEPEDFGLYYCQQH | | | |
| | KMNSLQTEDTSTYYCARDRD | | | | NEYPLTFGSGTKLEIK (SEQ ID | | | |
| | VLYYDGDFSPFVMDAWGQG | | | | NO: 146) | | | |
| | ASVTVSS (SEQ ID NO: 24) | | | | | | | |
| C4.1075 | EVQLKESGPGLVQPSQTLSLT | SSNIN | AISSGGSAFY | GGSEDPDY | DIVMTQSPSSLAVSAGETVTINC | KSSQSLLYS | WSSTRQS | QQYYDTPL |
| | CTVSGLSLISSNINWVRQPPG | (SEQ ID | NSALKS | FDY (SEQ | KSSQSLLYSGNQKNYLAWYQQ | GNQKNYL | (SEQ ID | T (SEQ ID |
| | KGLEWIGAISSGGSAFYNSAL | NO: 56) | (SEQ ID | ID NO: 110) | KPGQSPKLLIYWSSTRQSGVPD | A (SEQ ID | NO: 198) | NO: 221) |
| | KSRLTSSRDTSKSQVFLKMNS | | NO: 81) | | RFMGSGSGTDFTLTISSVQAED | NO: 172) | | |
| | LQTEDTAIYFCARGGSEDPDY | | | | LAIYYCQQYYDTPLTFGSGTKLEI | | | |
| | FDYWGQGVMVTVSS (SEQ | | | | K (SEQ ID NO: 141) | | | |
| | ID NO: 25) | | | | | | | |
| C4.1078 | EVQLVESGGGLVQPGGSLRLS | DFYMS | VIRNKAYVYT | NWEGFAS | DVVMTQTPPSLSVAIGQSVSISC | KSSQSLVY | RVSNLDS | AQTTHFPW |
| | CAASGFTFTDFYMSWIRQPP | (SEQ ID | TEYNPSVKG | (SEQ ID | KSSQSLVYSDGKTYLHWLLENS | SDGKTYLH | (SEQ ID | T (SEQ ID |
| | GKTPEWLGVIRNKAYVYTTEY | NO: 57) | (SEQ ID | NO: 115) | GRSPKRLIYRVSNLDSGVPDRFS | (SEQ ID | NO: 201) | NO: 226) |
| | NPSVKGRFTISRDNTQNILYL | | NO: 82) | | GTGSQKDFTLKISRVEAQDLGV | NO: 159) | | |
| | QMNTLRVEDTAIYYCARNWE | | | | YYCAQTTHFPWTFGGGTKLELK | | | |
| | GFASWGQGTLVTVSS (SEQ | | | | (SEQ ID NO: 147) | | | |
| | ID NO: 26) | | | | | | | |
| C4.1131 | EVKLVESDGGLVQPRKSLKLS | DYYMA | TISNDGSTKY | LTIRYKGVM | DVVMTQSPSHLAASPGESVSIN | KASKNIYK | SGSTLQS | QHHNEYPP |
| | CAASGFTFTDYYMAWVRQG | (SEQ ID | YRDSVKG | DA (SEQ ID | CKASKNIYKYLAWYQQKPGKTN | YLA (SEQ | (SEQ ID | T (SEQ ID |
| | PTMGLEWVATISNDGSTKYY | NO: 39) | (SEQ ID | NO: 116) | KLLIYSGSTLQSGTPSRFSGSGSG | ID | NO: 192) | NO: 216) |
| | RDSVKGRFTLSRDNAKTTLHL | | NO: 83) | | SDFTLTIRNLEPEDFGLYYCQHH | NO: 176) | | |
| | QMDSLRSEDTATYYCARLTIR | | | | NEYPPTFGGGTKLEIK (SEQ ID | | | |
| | YKGVMDAWGQGASVTVSS | | | | NO: 148) | | | |
| | (SEQ ID NO: 27) | | | | | | | |
| C4.1134 | EVQLVESDGGLVQPGRSLKLS | DFYMA | TISHDGRNTF | LTTRYKGV | DVVMTQSPSHLAASPGESVSIN | KASKNIYK | SGSTLQS | QHHNEYPP |
| | CAASGFTFSDFYMAWVRQA | (SEQ ID | YRDSVKG | MEA (SEQ | CKASKNIYKYLAWYQQKPGKTN | YLA (SEQ | (SEQ ID | T (SEQ ID |
| | PTKGLEWIATISHDGRNTFYR | NO: 58) | (SEQ ID | ID NO: 117) | KLLIYSGSTLQSGTPSRFSGSGSG | ID | NO: 192) | NO: 216) |
| | DSVKGRFTISRDNAKRALYLQ | | NO: 84) | | SDFTLTIRNLEPEDFGLYYCQHH | NO: 176) | | |
| | MDSLRSEDTATYYCASLTTRY | | | | NEYPPTFGGGTKLEIK (SEQ ID | | | |
| | KGVMEAWGQGASVTVSS | | | | NO: 148) | | | |
| | (SEQ ID NO: 28) | | | | | | | |
| C4.1138 | EVQLKESGPGLVKPSETLSLTC | SYHVS | VIWGDGNT | HPYYSIPY | DIVLTQSPTTMAAFPGEKVTITC | RASSSVSY | ETSKLAS | HQWSSIPLT |
| | TVSGFSLTSYHVSWVRQPPG | (SEQ ID | AYNSALKS | (SEQ ID | RASSSVSYMYWYQQKSGASPK | MY (SEQ | (SEQ ID | (SEQ ID |
| | KGLEWMGVIWGDGNTAYN | NO: 59) | (SEQ ID | NO: 118) | SWIYETSKLASGVPDRFSGSGSG | ID | NO: 202) | NO: 227) |
| | SALKSRLSINRDTSKSQVFLK | | NO: 85) | | TSYSFTISSMETEDAATYYCHQ | NO: 177) | | |
| | MNSLQTEDTATYYCARHPYY | | | | WSSIPLTFGSGTKLEIK (SEQ ID | | | |
| | SIPYWGQGASVTVSS (SEQ | | | | NO: 149) | | | |
| | ID NO: 29) | | | | | | | |
| C4.1139 | EVQLVESDGGLVQPGRSLKLS | DYYMA | SISYDGSTTY | LSIRYKGVM | DVVMTQSPSNLAASPGESVSIN | KASKSIGK | SGSTLQS | QHHIEYPPT |
| | CAASGFTFSDYYMAWVRQA | (SEQ ID | YRDSVKG | DA (SEQ ID | CKASKSIGKFLAWYQQKPGRPN | FLA (SEQ | (SEQ ID | (SEQ ID |
| | PTKGLYWVASISYDGSTTYYR | NO: 39) | (SEQ ID | NO: 119) | KLLIYSGSTLQSGTPSRFSGSGSG | ID | NO: 192) | NO: 228) |
| | DSVKGRFTISRDNAKSTLYLQ | | NO: 86) | | TDFTLTIRNLEPGDFGLYYCQHH | NO: 178) | | |
| | MGSLRSEDTATYYCARLSIRY | | | | IEYPPTFGPGTKLELK (SEQ ID | | | |
| | KGVMDAWGRGASVTVSS | | | | NO: 150) | | | |
| | (SEQ ID NO: 30) | | | | | | | |
| C4.1143 | EVQLVESDGGLLQPGRSLKLS | DFYMA | TISHDGRNT | LTTRYKGV | DVVMTQSPSHLAASPGESVSIN | KASKNIYK | SGSTLQS | QHHNEYPP |
| | CAASGFTFSDFYMAWVRQA | (SEQ ID | YYRDSVKG | MEA (SEQ | CKASKNIYKYLAWYQQKPGKTN | YLA (SEQ | (SEQ ID | T (SEQ ID |
| | PTKGLEWIATISHDGRNTYYR | NO: 58) | (SEQ ID | ID NO: 117) | KLLIYSGSTLQSGTPSRFSGSGSG | ID | NO: 192) | NO: 216) |
| | DSVKGRFTISRDNAKRTLYLQ | | NO: 87) | | SDFTLTIRNLEPEDFGLYYCQHH | NO: 176) | | |
| | MDSLRSEDTATYYCATLTTRY | | | | NEYPPTFGGGTKLEIK (SEQ ID | | | |
| | KGVMEAWGQGASVTVSS | | | | NO: 148) | | | |
| | (SEQ ID NO: 31) | | | | | | | |
| C4.1145 | EVKLVESGGGLVQPGRSLKLS | NYDMA | SIIPSGGTSYY | LTTSHYYV | DIWMTQSPTSMSISVGDRVTM | KASQNVG | KASNRYT | MQSNSYP |
| | CAASGFTFSNYDMAWVRQA | (SEQ ID | RDSVKG | MDA (SEQ | NCKASQNVGSNVDWYQQKTG | SNVD | (SEQ ID | WT (SEQ ID |
| | PTKGLEWVASIIPSGGTSYYR | NO: 60) | (SEQ ID | ID NO: 120) | QSPKLLIYKASNRYTGVPDRFTG | (SEQ ID | NO: 203) | NO: 229) |
| | DSVKGRFTVSRDNAKSTLYLQ | | NO: 88) | | SGSGTDFTFTISNMQAEDLAVY | NO: 179) | | |
| | MDSLRSEDTATYYCARLTTSH | | | | YCMQSNSYPWTFGGGTKLELK | | | |
| | YYVMDAWGQGASVTVSS | | | | (SEQ ID NO: 151) | | | |
| | (SEQ ID NO: 32) | | | | | | | |
| C4.1146 | EVKLVESGGGLVQPGRSLKLS | NYDMA | SIIPSGGTSYY | LTTSHYYV | DIWMTKSPSSMSASLGDRVTIT | QASQDIG | YATNLAN | LQYKQYPY |
| | CAASGFTFSNYDMAWVRQA | (SEQ ID | RDSVKG | MDA (SEQ | CQASQDIGNNLIWFQQKPGKS | NNLI (SEQ | (SEQ ID | T (SEQ ID |
| | PTKGLEWVASIIPSGGTSYYR | NO: 60) | (SEQ ID | ID NO: 120) | PRRMIYYATNLANGVPSRFSGS | ID | NO: 204) | NO: 230) |
| | DSVKGRFTVSRDNAKSTLYLQ | | NO: 88) | | RSGSDYSLTIISLESEDMADYHC | NO: 180) | | |
| | MDSLRSEDTATYYCARLTTSH | | | | LQYKQYPYTFGAGTKLEIK (SEQ | | | |
| | YYVMDAWGQGASVTVSS | | | | ID NO: 152) | | | |
| | (SEQ ID NO: 32) | | | | | | | |
| C4.1148 | EVQLVESDGGLVQPGRSLKLS | DYYMA | TISYDAFTTY | LSTRYKGV | DIQMTQSPSNLAASPGESVSIN | KASETIYKY | SGSTLQS | QHHVEYPP |
| | CAASGFTFSDYYMAWVRQA | (SEQ ID | YRDSVKG | MDA (SEQ | CKASETIYKYLAWYQQKPGRTN | LA (SEQ ID | (SEQ ID | T (SEQ ID |
| | PPKGLEWVATISYDAFTTYYR | NO: 39) | (SEQ ID | ID NO: 121) | KLLIYSGSTLQSGTPSRFSGSGSG | NO: 181) | NO: 192) | NO: 231) |
| | DSVKGRFTISRDNAKTILFLQ | | NO: 89) | | TDFTLTIRNLEPEDFGRYYCQHH | | | |
| | MDSLRSEDTATYYCIRLSTRYK | | | | VEYPPTFGAGTKLELK (SEQ ID | | | |
| | GVMDAWGHGASVTVSS | | | | NO: 153) | | | |
| | (SEQ ID NO: 33) | | | | | | | |
| C4.1152 | EVKLVESDGGLVQPGRSLKLS | DFYMA | SISHDGRNTY | LTARYKGV | DVVMTQSPSHLAASPGESVSIN | KASKNIYK | SGSTLQS | SGSTLQS |
| | CAASGFTFSDFYMAWVRQA | (SEQ ID | YRDSVKG | MGA (SEQ | CKASKNIYKYLAWYQQKPGKTN | YLA (SEQ | (SEQ ID | (SEQ ID |
| | PTKGLEWIASISHDGRNTYYR | NO: 58) | (SEQ ID | ID NO: 122) | KLLIYSGSTLQSGTPSRFSGSGSG | ID | NO: 192) | NO: 192) |
| | DSVKGRFTISRDNAKRTLYLQ | | NO: 90) | | SDFTLTIRNLEPEDFGLYYCQHH | NO: 176) | | |
| | MDSLRSEDTATYYCASLTARY | | | | NEYPPTFGGGTKLEIK (SEQ ID | | | |
| | KGVMGAWGQGTLVTVSS | | | | NO: 148) | | | |
| | (SEQ ID NO: 34) | | | | | | | |
| C4.1155 | EVKLLESGGGLVQPGGSLRLS | DFYMS | VIRNRAIGFR | NNLGFDY | TPPSLSVAIGQSVSISCKSSQSLV | KSSQSLVY | QVSNLDS | AQTTHFPY |
| | CATSGFSFTDFYMSWIRQPL | (SEQ ID | TEYNPSVKG | (SEQ ID | YSDGKTYLHWLLQISGRSPERLI | SDGKTYLH | (SEQ ID | T (SEQ ID |
| | GKAPEWLGVIRNRAIGFRTEY | NO: 57) | (SEQ ID | NO: 123) | YQVSNLDSGVPDRFSGTGSQK | (SEQ ID | NO: 186) | NO: 232) |
| | NPSVKGRFTISRDNTQNILYL | | NO: 91) | | DFTLKISRVEAKDLGFYYCAQTT | NO: 159) | | |
| | QMNTLRTEDTAIYYCARNNL | | | | HFPYTFGAGTKLELK (SEQ ID | | | |
| | GFDYWGQGASVTVSS (SEQ | | | | NO: 154) | | | |
| | ID NO: 35) | | | | | | | |
| C4.1164 | EVQLVESDGGLVQPGRSLKLS | DYYMA | SISYDGSTTY | LSIRYKGVM | DIQMTQSPSNLAASPGESVSIN | KASETIYKY | SGSTLQS | QHHVEYPP |
| | CAASGFTFSDYYMAWVRQA | (SEQ ID | YRDSVKG | DA (SEQ ID | CKASETIYKYLAWYQQKPGRTN | LA (SEQ ID | (SEQ ID | T (SEQ ID |
| | PTKGLYWVASISYDGSTTYYR | NO: 39) | (SEQ ID | NO: 119) | KLLIYSGSTLQSGTPSRFSGSGSG | NO: 181) | NO: 192) | NO: 231) |
| | DSVKGRFTISRDNAKSTLYLQ | | NO: 86) | | TDFTLTIRNLEPEDFGRYYCQHH | | | |
| | MGSLRSEDTATYYCARLSIRY | | | | VEYPPTFGAGTKLELK (SEQ ID | | | |
| | KGVMDAWGRGASVTVS | | | | NO: 153) | | | |
| | (SEQ ID NO: 36) | | | | | | | |
| C4.947 | EVKLVESGGGLVQPGGSMRL | DFYMN | FIRNKANAYT | SNYGFDY | DVAMTQTPPSLSVAIGQSVSISC | KSSQSLVY | QVSNLDS | AQTTHFPFT |
| | SCAASGFTFTDFYMNWIRQP | (SEQ ID | TAYNPSVKG | (SEQ ID | KSSQSLVYSDGKTYLHWLLQSS | SDGKTYLH | (SEQ ID | (SEQ ID |
| | AGKAPEWLGFIRNKANAYTT | NO: 41) | (SEQ ID | NO: 96) | GRSPKRLIYQVSNLDSGVPDRFS | (SEQ ID | NO: 186) | NO: 209) |
| | AYNPSVKGRFTISRDNAQNM | | NO: 64) | | GTGSQKDFTLKISRVEAKDLGVY | NO: 159) | | |
| | LYLQMNTLRAEDTATYYCARS | | | | YCAQTTHFPFTFGSGTKLEIK | | | |
| | NYGFDYWGQGVMVTVSS | | | | (SEQ ID NO: 128) | | | |
| | (SEQ ID NO: 5) | | | | | | | |

The anti-ABCC4 antibodies listed in Table 2 are also referred to as anti-KPC4 antibodies or anti-C4 antibodies and can be referred to by the antibody number listed in Table 2.

In some embodiments, the antibody comprises a VL region and a VH region that are present in separate polypeptides; in other embodiments, the VL region and a VH region are contained within a single polypeptide.

The antibody of the present disclosure may be selected from the group consisting of an Ig monomer, a Fab fragment, a F(ab)₂ fragment, a Fd fragment, a scFv, a scAb, a dAb, and a Fv.

In some embodiments, a subject antibody is a recombinant or modified antibody, e.g., a chimeric, humanized, deimmunized or an in vitro generated antibody. The term “recombinant” or “modified” antibody as used herein is intended to include all antibodies that are prepared, expressed, created, or isolated by recombinant means, such as (i) antibodies expressed using a recombinant expression vector transfected into a host cell; (ii) antibodies isolated from a recombinant, combinatorial antibody library; (iii) antibodies isolated from an animal (e.g. a mouse) that is transgenic for human immunoglobulin genes; or (iv) antibodies prepared, expressed, created, or isolated by any other means that involves splicing of human immunoglobulin gene sequences to other DNA sequences. Such recombinant antibodies include humanized, CDR grafted, chimeric, deimmunized, and in vitro generated antibodies; and can optionally include constant regions derived from human germline immunoglobulin sequences.

As noted above, the subject anti-ABCC4 antibody specifically binds one or more epitopes of ABCC4. Thus, the epitope is an ABCC4 epitope. The size of a ABCC4 epitope bound by anti-ABCC4 antibody may vary, including where the ABCC4 epitope is formed by a polypeptide having a contiguous stretch of an ABCC4 sequence that may range from 3 aa or less to 12 aa or more, including but not limited to e.g., 4 aa, 5 aa, 6 aa, 7 aa, 8 aa, 9 aa, 10 aa, 11 aa, 12 aa, 4 aa to 10 aa, 5 aa to 10 aa, 6 aa to 10 aa, 4 aa to 8 aa, 5 aa to 8 aa, 6 aa to 8 aa, etc.

In some embodiments, the ABCC4 epitope can be formed by a polypeptide having at least about 75%, at least about 80%, at least about 85%, at least about 90%, at least about 95%, at least about 98%, at least about 99%, or 100%, amino acid sequence identity to a contiguous stretch of a ABCC4 sequence, including but not limited to e.g., the human ABCC4 sequence: MLPVYQEVKPNPLQDANLCSRVFFVWVLNPLFKIGHKRRLEEDDMYSVLPEDRSQHLGEELQG FWDKEVLRAENDAQKPSLTRAIIKCYWKSYLVLGIFTLIEESAKVIQPIFLGKIINYFENYDPMDSV ALNTAYAYATVLTFCTLILAILHHLYFYHVQCAGMRLRVAMCHMIYRKALRLSNMAMGKTTTGQI VNLLSNDVNKFDQVTVFLHFLWAGPLQAIAVTALLVVMElGISCLAGMAVLIILLPLQSCFGKLFSS LRSKTATFTDARIRTMNEVITGIRIIKMYAWEKSFSNLITNLRKKEISKILRSSCLRGMNLASFFSA SKIIVFVTFTTYVLLGSVITASRVFVAVTLYGAVRLTVTLFFPSAIERVSEAIVSIRRIQTFLLLDEIS QRNRQLPSDGKKMVHVQDFTAFVVDKASETPTLQGLSFTVRPGELLAVVGPVGAGKSSLLSAV LGELAPSHGLVSVHGRIAYVSQQPVVVFSGTLRSNILFGKKYEKERYEKVIKACALKKDLQLLED GDLTVIGDRGTTLSGGQKARVNLARAVYQDADIYLLDDPLSAVDAEVSRHLFELCICQILHEKITI LVTHQLQYLKAASQILILKDGKMVQKGTYTEFLKSGIDFGSLLKKDNEESEQPPVPGTPTLRNRT FSESSVVVSQQSSRPSLKDGALESQDTENVPVTLSEENRSEGKVGFQAYKNYFRAGAHVVIVFIF LILLNTAAQVAYVLQDVVVVLSYVVANKQSMLNVTVNGGGNVTEKLDLNVVYLGIYSGLTVATVLFG IARSLLVFYVLVNSSQTLHNKMFESILKAPVLFFDRNPIGRILNRFSKDIGHLDDLLPLTFLDFIQTL LQVVGVVSVAVAVIPVVIAIPLVPLGIIFlFLRRYFLETSRDVKRLESTTRSPVFSHLSSSLQGLVVTI RAYKAEERCQELFDAHQDLHSEAVVFLFLTTSRVVFAVRLDAICAMFVLIVAFGSLILAKTLDAGQV GLALSYALTLMGMFQVVCVRQSAEVENMMISVERVIEYTDLEKEAPVVEYQKRPPPAVVPHEGVII FDNVNFMYSPGGPLVLKHLTALIKSQEKVGIVGRTGAGKSSLISALFRLSEPEGKIVVIDKILTTEIG LHDLRKKMSIIPQEPVLFTGRKNLDPFNEHTDEELVVNALQEVQLKETIEDLPGKMDTELAES GSNFSVGQRQLVCLARAILRKNQILIIDEATANVDPRTDELIQKKIREKFAHCTVLTIAHRLNTIIDS DKIMVLDSGRLKEYDEPYVLLQNKESLFYKMVQQLGKAEAAALTETAKQVYFKRNYPHIGHTD HMVTNTSNGQPSTLTIFETAL (SEQ ID NO:233), e.g., an extracellular region thereof.

A subject anti-ABCC4 antibody exhibits high affinity binding to ABCC4. For example, a subject anti-ABCC4 antibody may bind to a human ABCC4 with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M. A subject anti-ABCC4 antibody may bind to an epitope present on ABCC4 with an affinity of from about 10⁻⁷ M to about 10⁻⁸ M, from about 10⁻⁸ M to about 10⁻⁹ M, from about 10⁻⁹ M to about 10⁻¹⁰ M, from about 10⁻¹⁰ M to about 10⁻¹¹ M, or from about 10⁻¹¹ M to about 10⁻¹² M, or greater than 10⁻¹² M.

A subject anti-ABCC4 antibody exhibits substantially no binding to any epitopes formed by amino acids within other related, but sequence dissimilar, proteins such as related but sequence dissimilar EPs. Any binding of a subject anti-ABCC4 antibody to an epitope formed by amino acids within a related, but sequence dissimilar, protein is generally non-specific binding of a substantially lower affinity than the specific binding of the anti-ABCC4 antibody to the epitope on ABCC4. A substantially lower affinity is generally at least a 2 fold, 3 fold, 5 fold, 10 fold, 50 fold, 100 fold, 500 fold, or 1000 fold lower affinity.

A subject anti-ABCC4 antibody can reduce transport of molecules through an ABCC4 transporter, e.g., a human ABCC4. For example, a subject anti-ABCC4 antibody can reduce transport by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the degree of transport in the absence of the anti-ABCC4 antibody.

In some embodiments, a subject antibody comprises FR regions that are mammalian sequences, including e.g., rodent, non-human primate, and human sequences (e.g., encoded by the respective heavy chain FR-encoding sequences).

A subject antibody can comprise a heavy chain variable (VH) region comprising an amino acid sequence that is 85%, 86%, 87%, 88%, 89%, 90%, 91%, 92%, 93%, 94%, 95%, 96%, 97%, 98%, 99%, or more, including 100%, identical to a sequence for a VH region of a VH-VL pair of an antibody set forth in Table 2. The subject antibody can comprise a light chain variable (VL) region comprising an amino acid sequence that is 85%, 86%, 87%, 88%, 89%, 90%, 91%, 92%, 93%, 94%, 95%, 96%, 97%, 98%, 99%, or more, including 100%, identical to a sequence for a VL of the VH-VL region pair of the antibody set forth in Table 2.

Regions and/or chains of the subject antibodies may or may not be joined by one or more linker regions. Where present, the linker region can be from about 5 amino acids to about 50 amino acids in length, e.g., from about 5 aa to about 10 aa, from about 10 aa to about 15 aa, from about 15 aa to about 20 aa, from about 20 aa to about 25 aa, from about 25 aa to about 30 aa, from about 30 aa to about 35 aa, from about 35 aa to about 40 aa, from about 40 aa to about 45 aa, or from about 45 aa to about 50 aa in length.

Linkers suitable for use a subject antibody include “flexible linkers”. If present, the linker molecules are generally of sufficient length to permit some flexible movement between linked regions. The linker molecules are generally about 6-50 atoms long. The linker molecules may also be, for example, aryl acetylene, ethylene glycol oligomers containing 2-10 monomer units, diamines, diacids, amino acids, or combinations thereof. Other linker molecules which can bind to polypeptides may be used in light of this disclosure.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, GSGGSₙ (SEQ ID NO:234) and GGGSₙ (SEQ ID NO:235), where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited to GGSG (SEQ ID NO:236), GGSGG (SEQ ID NO:237), GSGSG (SEQ ID NO:238), GSGGG (SEQ ID NO:239), GGGSG (SEQ ID NO:240), GSSSG (SEQ ID NO:241), and the like. The ordinarily skilled artisan will recognize that design of a peptide conjugated to any elements described above can include linkers that are all or partially flexible, such that the linker can include a flexible linker as well as one or more portions that confer less flexible structure.

In other instances, the flexibility of the hinge region of an antibody of the present disclosure may be reduced by either mutating amino acid C220 to serine or any other natural amino acid, by removing C220, by removing the complete hinge, or by replacing the IgG1 hinge with an IgG3 hinge, an antibody is formed in which the light chains are connected via their C-terminal cysteines, analogous to the situation found in the human isotype IgA2m. This results in a reduced flexibility of the Fabs relative to the Fc and consequently reduced cross-linking capacity. Another strategy to reduce the flexibility of an IgG1 molecule is to replace the IgG1 hinge with the IgG2 hinge or IgG2-like hinge. Alternatively, a variant of the IgG1 hinge that resembles the IgG2 hinge can be introduced. This mutant (TH7Δ6-9) contains mutation T223C and two deletions (K222 and T225) in order to create a shorter hinge with an additional cysteine.

The substitution of mouse or rat CDRs into a human variable domain framework can result in retention of their correct spatial orientation where, e.g., the human variable domain framework adopts the same or similar conformation to the mouse or rat variable framework from which the CDRs originated. This can be achieved by obtaining the human variable domains from human antibodies whose framework sequences exhibit a high degree of sequence identity with the murine variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies. See Kettleborough et al., Protein Engineering 4:773 (1991); Kolbinger et al., Protein Engineering 6:971 (1993).

Having identified the complementarity determining regions of the murine donor immunoglobulin and appropriate human acceptor immunoglobulins, the next step is to determine which, if any, residues from these components should be substituted to optimize the properties of the resulting humanized antibody. In general, substitution of human amino acid residues with murine should be minimized, because introduction of murine residues increases the risk of the antibody eliciting a human-anti-mouse-antibody (HAMA) response in humans. Art-recognized methods of determining immune response can be performed to monitor a HAMA response in a particular patient or during clinical trials. Patients administered humanized antibodies can be given an immunogenicity assessment at the beginning and throughout the administration of said therapy. The HAMA response is measured, for example, by detecting antibodies to the humanized therapeutic reagent, in serum samples from the patient using a method known to one in the art, including surface plasmon resonance technology (BIACORE) and/or solid-phase ELISA analysis. In many embodiments, a subject humanized antibody does not substantially elicit a HAMA response in a human subject.

Certain amino acids from the human variable region framework residues are selected for substitution based on their possible influence on CDR conformation and/or binding to antigen. The unnatural juxtaposition of murine CDR regions with human variable framework region can result in conformational restraints, which, unless corrected by substitution of certain amino acid residues, lead to loss of binding affinity.

The selection of amino acid residues for substitution can be determined, in part, by computer modeling. Computer hardware and software for producing three-dimensional images of immunoglobulin molecules are known in the art. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, and preferably those sharing at least 60%, 70%, 80%, 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

In some embodiments, a subject antibody comprises scFv multimers. For example, in some embodiments, a subject antibody is an scFv dimer (e.g., comprises two tandem scFv (scFv₂)), an scFv trimer (e.g., comprises three tandem scFv (scFv₃)), an scFv tetramer (e.g., comprises four tandem scFv (scFv₄)), or is a multimer of more than four scFv (e.g., in tandem). The scFv monomers can be linked in tandem via linkers of from about 2 amino acids to about 15 amino acids in length, e.g., 2 aa, 3 aa, 4 aa, 5 aa, 6 aa, 7 aa, 8 aa, 9 aa, 10 aa, 11 aa, 12 aa, 13 aa, 14 aa, or 15 aa in length. Suitable linkers include, e.g., (Gly)ₓ, where x is an integer from 2 to Other suitable linkers are those discussed above. In some embodiments, each of the scFv monomers in a subject scFV multimer is humanized, as described above. In certain embodiments, a bispecific antibody may be in any molecular format known in the literature. For example, a bispecific antibody of the present disclosure may have a molecular format described in Spiess C. et al., Mol Immunol. 2015 October; 67(2 Pt A): 95-106.

In some embodiments, a subject antibody comprises a constant region of an immunoglobulin (e.g., an Fc region). The Fc region, if present, can be a human Fc region. If constant regions are present, the antibody can contain both light chain and heavy chain constant regions. Suitable heavy chain constant region include CH1, hinge, CH2, CH3, and CH4 regions. The antibodies described herein include antibodies having all types of constant regions, including IgM, IgG, IgD, IgA and IgE, and any isotype, including IgG1, IgG2, IgG3 and IgG4. An example of a suitable heavy chain Fc region is a human isotype IgG1 Fc. Light chain constant regions can be lambda or kappa. A subject antibody (e.g., a subject humanized antibody) can comprise sequences from more than one class or isotype. Antibodies can be expressed as tetramers containing two light and two heavy chains, as separate heavy chains, light chains, as Fab, Fab′ F(ab′)2, and Fv, or as single chain antibodies in which heavy and light chain variable domains are linked through a spacer.

In some embodiments, a subject antibody comprises a free thiol (—SH) group at the carboxyl terminus, where the free thiol group can be used to attach the antibody to a second polypeptide (e.g., another antibody, including a subject antibody), a scaffold, a carrier, etc.

A subject antibody can be covalently linked to a second moiety (e.g., a lipid, a polypeptide other than a subject antibody, a synthetic polymer, a carbohydrate, a toxin and the like) using for example, glutaraldehyde, a homobifunctional cross-linker, or a heterobifunctional cross-linker.

Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a one-step reaction procedure in which the cross-linker is added to a solution containing a mixture of the polypeptides to be linked. Homobifunctional NHS ester and imido esters cross-link amine containing polypeptides. In a mild alkaline pH, imido esters react only with primary amines to form imidoamides, and overall charge of the cross-linked polypeptides is not affected. Homobifunctional sulfhydryl reactive cross-linkers includes bismaleimidohexane (BMH), 1,5-difluoro-2,4-dinitrobenzene (DFDNB), and 1,4-Bis[3-(2-pyridyldithio)propionamido]butane (DPDPB).

Compositions and Formulations

The present disclosure provides a composition comprising a subject antibody. A subject antibody composition can comprise, in addition to a subject antibody, one or more of: a salt, e.g., NaCl, MgCl₂, KCl, MgSO₄, etc.; a buffering agent, e.g., a Tris buffer, a histidine buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-etc.; a protease inhibitor; glycerol; and the like.

Compositions of the present disclosure also include pharmaceutical compositions that include an antibody described herein. In general, a formulation comprises an effective amount of the subject antibody. An “effective amount” means a dosage sufficient to produce a desired result, e.g., reduction in a cancer of a subject, reduction in the growth rate of a cancer in a subject, amelioration of a symptom of cancer, and the like. Generally, the desired result is at least a reduction in a symptom of a cancer, reduction in the growth of a cancer, reduction in the size of a cancer, etc., as compared to a control. A subject antibody can be delivered, or be formulated, in such a manner as to avoid the blood-brain barrier.

In some instances, an antibody may include a delivery enhancer, including where such enhancers may facilitate crossing of the blood-brain barrier, increased permeability, e.g., allowing for efficient transdermal delivery, and the like.

In some instances, the antibodies of the present disclosure may not be administered in a formulation with a delivery enhancer. In some instances, the antibodies of the present disclosure may themselves enhance permeability across the blood-brain barrier. In some instances, the antibodies of the present disclosure may be used as a delivery enhancer to facilitate crossing of the blood-brain barrier by an anti-neoplastic agent, e.g., an immunotherapeutic agent or a chemotherapeutic agent. In some instances, the antibodies of the present disclosure may be used as a delivery enhancer to facilitate crossing of the blood-brain barrier, blood-cerebrospinal fluid (CSF) barrier, blood-testis barrier, or blood-placenta barrier by an active agent, such as, another antibody or a chemotherapeutic agent.

In the subject methods, a subject antibody can be administered to the host using any convenient means capable of resulting in the desired therapeutic effect or diagnostic effect. Thus, the agent can be incorporated into a variety of formulations for therapeutic administration. More particularly, a subject antibody can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, a subject antibody can be administered in conjunction with a pharmaceutically acceptable excipient, or they may also be used alone or in appropriate association, as well as in combination, with other pharmaceutically active compounds. The following methods and excipients are merely exemplary and are in no way limiting.

A subject antibody can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions comprising a subject antibody are prepared by mixing the antibody having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration.

Exemplary antibody concentrations in a subject pharmaceutical composition may range from about 1 mg/mL to about 200 mg/ml or from about 50 mg/mL to about 200 mg/mL, or from about 150 mg/mL to about 200 mg/mL.

An aqueous formulation of the antibody may be prepared in a pH-buffered solution, e.g., at pH ranging from about 4.0 to about 7.5 or from about 5.0 to about 6.0, or alternatively about 5.5 Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 nM.

A surfactant may also be added to the antibody formulation to reduce aggregation of the formulated antibody and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Exemplary concentrations of surfactant may range from about 0.001% to about 1% w/v.

A lyoprotectant may also be added in order to protect the labile active ingredient (e.g. a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a subject formulation includes a subject antibody, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

For example, a subject formulation can be a liquid or lyophilized formulation suitable for parenteral administration, and can comprise: about 1 mg/mL to about 200 mg/mL of a subject antibody; about 0.001% to about 1% of at least one surfactant; about 1 mM to about 100 mM of a buffer; optionally about 10 mM to about 500 mM of a stabilizer; and about 5 mM to about 305 mM of a tonicity agent; and has a pH of about 4.0 to about 7.0.

A subject antibody can be utilized in aerosol formulation to be administered via inhalation. A subject antibody can be formulated into pressurized acceptable propellants such as dichlorodifluoromethane, propane, nitrogen and the like.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for a subject antibody may depend on the particular antibody employed and the effect to be achieved, and the pharmacodynamics associated with each antibody in the host.

A subject antibody can be administered as an injectable formulation. Typically, injectable compositions are prepared as liquid solutions or suspensions; solid forms suitable for solution in, or suspension in, liquid vehicles prior to injection may also be prepared. The preparation may also be emulsified or the antibody encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, a subject antibody is formulated in a controlled release formulation. Sustained-release preparations may be prepared using methods well known in the art.

Dosages

A suitable dosage can be determined by an attending physician or by other qualified medical personnel, based on various clinical factors. As is well known in the medical arts, dosages for any one patient depend upon many factors, including the patient's size, body surface area, age, the particular compound to be administered, sex of the patient, time, and route of administration, general health, and other drugs being administered concurrently. A subject antibody may be administered in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 pg to 10 mg per kilogram of body weight per minute.

Those of skill will readily appreciate that dose levels can vary as a function of the specific antibody, the severity of the symptoms and the susceptibility of the subject to side effects.

Preferred dosages for a given compound are readily determinable by those of skill in the art by a variety of means.

Routes of Administration

A subject antibody is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, subcutaneous, intradermal, topical application, intravenous, intraarterial, rectal, nasal, oral, and other enteral and parenteral routes of administration. Routes of administration may be combined, if desired, or adjusted depending upon the antibody and/or the desired effect. A subject antibody composition can be administered in a single dose or in multiple doses. In some embodiments, a subject antibody composition is administered orally. In some embodiments, a subject antibody composition is administered via an inhalational route. In some embodiments, a subject antibody composition is administered intranasally. In some embodiments, a subject antibody composition is administered locally. In some embodiments, a subject antibody composition is administered intracranially. In some embodiments, a subject antibody composition is administered intravenously.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of a subject antibody. Where systemic delivery is desired, administration typically involves invasive or systemically absorbed topical or mucosal administration of pharmaceutical preparations.

A subject antibody can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as cancer and/or the growth of a cancer and pain associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of subjects (wherein the term “subject” is used interchangeably herein with the terms “individual” and “patient”) are treatable according to the presently disclosed methods. Generally, such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the hosts will be humans.

Kits with unit doses of a subject antibody, e.g. in oral or injectable doses, are provided. In some embodiments, in addition to the containers containing the unit doses will be an informational package insert describing the use and attendant benefits of the antibody in treating pathological condition of interest.

Nucleic Acids

The present disclosure provides nucleic acids comprising nucleotide sequences encoding a subject antibody. A nucleotide sequence encoding a subject antibody can be operably linked to one or more regulatory elements, such as a promoter and enhancer, that allow expression of the nucleotide sequence in the intended target cells (e.g., a cell that is genetically modified to synthesize and/or secrete the encoded antibody).

Suitable promoter and enhancer elements are known in the art. For expression in a bacterial cell, suitable promoters include, but are not limited to, lacl, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

A nucleotide sequence encoding a subject antibody can be present in an expression vector and/or a cloning vector. Where a subject antibody comprises two or more separate polypeptides, nucleotide sequences encoding the two polypeptides can be cloned in the same or separate vectors. Separate polypeptides may be expressed from a single nucleic acid or single vector using various strategies, such as separate promoters, one or more internal ribosomal entry sites (IRES), one or more self-cleaving sequences (e.g., 2A cleavage sequences, e.g., P2A, T2A, E2A, and F2A), combinations thereof, and the like. An expression vector can include a selectable marker, an origin of replication, and other features that provide for replication and/or maintenance of the vector.

Large numbers of suitable vectors and promoters are known to those of skill in the art; many are commercially available for generating a subject recombinant construct. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus; adeno-associated virus; SV40; herpes simplex virus; human immunodeficiency virus; a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Nucleic acids, e.g., as described herein, may, in some instances, be introduced into a cell, e.g., by contacting the cell with the nucleic acid. Cells with introduced nucleic acids will generally be referred to herein as genetically modified cells. Various methods of nucleic acid delivery may be employed including but not limited to e.g., naked nucleic acid delivery, viral delivery, chemical transfection, biolistics, and the like.

Cells

The present disclosure provides isolated genetically modified cells (e.g., in vitro cells, ex vivo cells, cultured cells, etc.) that are genetically modified with a subject nucleic acid. In some embodiments, a subject isolated genetically modified cell can produce a subject antibody. In some instances, a genetically modified cell can deliver an antibody, e.g., to a subject in need thereof.

Suitable cells include eukaryotic cells, such as a mammalian cell, an insect cell, a yeast cell; and prokaryotic cells, such as a bacterial cell. Introduction of a subject nucleic acid into the host cell can be affected, for example by calcium phosphate precipitation, DEAE dextran mediated transfection, liposome-mediated transfection, electroporation, or other known method.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells, CHO cells, 293 cells, 3T3 cells, Vero cells, Huh-7 cells, BHK cells, PC12 cells, COS cells, COS-7 cells, RAT1 cells, mouse L cells, human embryonic kidney (HEK) cells, HLHepG2 cells, and the like.

In some instances, useful mammalian cells may include cells derived from a mammalian tissue or organ. In some instances, cells employed are kidney cells, including e.g., kidney cells of an established kidney cell line, such as HEK 293T cells.

In some instances, cells of the present disclosure may be immune cells. As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), T-regulatory cells (Treg) and gamma-delta T cells. A “cytotoxic cell” includes CD8+ T cells, natural-killer (NK) cells, and neutrophils, which cells are capable of mediating cytotoxicity responses.

In some instances, useful cells expressing an antibody such as a multi-specific antibody of the present disclosure may include producer T cells. Producer T cells engineered to include nucleic acid sequence encoding an antibody of the present disclosure may, in some instances, be employed to deliver the antibody to a subject in need thereof.

In some instances, immune cells of the present disclosure include immune effector cell comprising a chimeric antigen receptor (CAR) comprising an ABCC4 binding domain, a transmembrane domain, and an intracellular signaling domain, and wherein the ABCC4 binding domain comprises heavy chain complementarity determining regions (HCDRs) and light chain CDRs (LCDRs) of a pair of variable heavy chain (VH) region and variable light chain (VL) region of an antibody listed in Table 2. In one embodiment, the intracellular signaling domain may include one or more functional signaling domains derived from at least one costimulatory molecule, e.g., 4-1BB (i.e., CD137), CD27 and/or CD28. The intracellular signaling domain may include a functional signaling domain derived from a costimulatory molecule and a functional signaling domain derived from a stimulatory molecule.

The immune effector cell may be a T-cell. The immune effector cell may be an autologous cell.

Methods

As summarized above, methods of the present disclosure include methods of contacting a cell with an antibody of the present disclosure, methods of treating a subject according to a method that involves administering to the subject an antibody of the present disclosure, methods of making elements described in the instant application, including e.g., antibodies, compositions and formulations, nucleic acids, expression vectors, cells, and the like.

As summarized above, methods of the present disclosure include contacting a cancer cell with an antibody of the present disclosure, e.g., to detect presence of expression of ABCC4 on the cancer cell, measure level of expression of ABCC4 on the cancer cell, or to facilitate and/or enhance killing of the cancer cell. In some instances, killing of the cancer cell is mediated by an immune response or immune cell acting upon the cancer cell bound by the antibody. In some instances, killing of the cancer cell is mediated by inhibition of cellular efflux of the cancer cell, e.g., as a result of ABCC4 inhibition by the antibody. In some instances, killing of the cancer cell is mediated by a combination of inhibition of cellular efflux of the cancer cell plus an immune mediated response (e.g., via Fc region of the antibody). Methods that involve contacting a cancer cell with an antibody of the present disclosure may or may not include contacting the cancer cell with an additional therapy or active agent, including e.g., a chemotherapeutic, an immunotherapy, radiation therapy, or the like.

Treatment Methods

The present disclosure provides methods of treating a cancer, the methods generally involving administering to an individual in need thereof (e.g., an individual having a cancer) an effective amount of an antibody as provided herein, alone (e.g., in monotherapy) or in combination (e.g., in combination therapy) with one or more additional therapeutic agents. Administration of an antibody of the present disclosure may be performed by any convenient and appropriate route of delivery.

Accordingly, administration includes but is not limited to e.g., delivery of the antibody by injection, delivery of the antibody by infusion, delivery of a nucleic acid or expression vector encoding the antibody, delivery of the antibody by administering to the subject a cell that expresses and secretes the antibody, delivery of an immune effector cell (e.g., a CAR-T cell) that expresses on the cell surface a chimeric antigen receptor (CAR) comprising a ABCC4 binding domain, a transmembrane domain, and an intracellular signaling domain, and wherein the ABCC4 binding domain comprises HCDRs and LCDRs of a pair of VH region and VL region of an antibody listed in Table 2, and the like. Administration of an agent, a nucleic acid encoding an agent, a cell expressing an agent, etc. may include contacting with the agent, contacting with the nucleic acid, contacting with the cell, and the like.

In some embodiments, an effective amount of a subject antibody is an amount that, when administered alone (e.g., in monotherapy) or in combination (e.g., in combination therapy) with one or more additional therapeutic agents, in one or more doses, is effective to reduce an adverse symptom of cancer by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the severity of the adverse symptom in the absence of treatment with the antibody.

In some embodiments, an effective amount of a subject antibody is an amount that, when administered alone (e.g., in monotherapy) or in combination (e.g., in combination therapy) with one or more additional therapeutic agents, in one or more doses, is effective to improve the cancer (i.e., slow the growth of the cancer, stop the growth of the cancer, reverse the growth of the cancer, kill cancer cells (including tumor cells, or the like) in the individual being treated. For example, an effective amount of a subject antibody can reduce a cancer growth rate or reduce a cancer size in an individual by at least about 5%, at least about 10%, at least about 15%, at least about 20%, at least about 25%, at least about 30%, at least about 40%, at least about 50%, or more, compared to in the absence of treatment with an antibody.

In some instances, a subject may be treated systemically, including with the subject antibody, with or without one or more additional reagents. By “systemic treatment”, as used herein, is meant a treatment that is not directed solely to target a specific tumor (such as e.g., a primary tumor or a defined secondary tumor) or a specific cancer containing tissue (such as e.g., the liver in the case of liver cancer, the blood in the case of a blood cancer, etc.). Systemic treatments will generally be directed to the subject's body as a whole and may include but are not limited to e.g., systemic radiation therapy, systemic chemotherapy, systemic immunotherapy, combinations thereof and the like.

In some instances, a subject may be treated locally, including with the subject antibody, with or without one or more additional reagents. By “local treatment”, as used herein, is meant a treatment that is specifically directed to the location of a tumor (such as e.g., a primary tumor or a defined secondary tumor) or specifically directed to a cancer containing tissue (such as e.g., the liver in the case of liver cancer, the blood in the case of a blood cancer, etc.). In some instances, local treatment may also be administered in such a way as to affect the environment surrounding a tumor, such as tissue surrounding the tumor, such as tissue immediately adjacent to the tumor. Local treatment will generally not affect or not be targeted to tissues distant from the site of cancer including the site of a tumor, such as a primary tumor. Useful local treatments that may be administered in addition to or in combination with a subject antibody, e.g., include but are not limited to surgery, local radiation therapy, local cryotherapy, local laser therapy, local topical therapy, combinations thereof, and the like.

In some embodiments, a subject treatment method involves administering a subject antibody and one or more additional therapeutic agents. Suitable additional therapeutic agents include, but are not limited to, chemotherapeutic agents, radiation therapy reagents, immunotherapy reagents, other antibody agents, and the like. Additional therapies that may be administered to a subject before, during or after a subject administering an antibody of the present disclosure will vary depending on numerous factors including e.g., the type of cancer, the subject's medical history, general state of health and/or any co-morbidities, and the like. Useful cancer therapies include but are not limited to e.g., radiation therapy, chemotherapy, immunotherapy, and the like.

Radiation therapy includes, but is not limited to, x-rays or gamma rays that are delivered from either an externally applied source such as a beam, or by implantation of small radioactive sources.

Suitable antibodies for use in cancer treatment include, but are not limited to, naked antibodies, e.g., trastuzumab (Herceptin), bevacizumab (Avastin™), cetuximab (Erbitux™) panitumumab (Vectibix™), Ipilimumab (Yervoy™), rituximab (Rituxan), alemtuzumab (Lemtrada™), Ofatumumab (Arzerra™), Oregovomab (OvaRex™), Lambrolizumab (MK-3475), pertuzumab (Perjeta™), ranibizumab (Lucentis™) etc., and conjugated antibodies, e.g., gemtuzumab ozogamicin (Mylortarg™), Brentuximab vedotin (Adcetris™), 90Y-labelled ibritumomab tiuxetan (Zevalin™), 131I-labelled tositumoma (Bexxar™), etc.

Suitable antibodies for use in cancer treatment also include, but are not limited to, antibodies raised against tumor-associated antigens. Such antigens include, but are not limited to, CD20, CD30, CD33, CD52, EpCAM, CEA, gpA33, Mucins, TAG-72, CAIX, PSMA, Folate-binding protein, Gangliosides (e.g., GD2, GD3, GM2, etc.), Ley, VEGF, VEGFR, Integrin alpha-V-beta-3, Integrin alpha-5-beta-1, EGFR, ERBB2, ERBB3, MET, IGF1R, EPHA3, TRAILR1, TRAILR2, RANKL, FAP, Tenascin, Programmed Death-Ligand 1 (PD-L1), androgen receptor (AR), Bruton's Tyrosine Kinase (BTK), BCR-Abl, c-kit, PIK3CA, EML4-ALK, KRAS, ALK, ROS1, AKT1, BRAF, MEKJ, MEK2, NRAS, RAC1, ESR1, CTLA-4, LAG-3 and TIM-3, etc. These antibodies may be administered as a combination therapy with an anti-ABCC4 antibody provided herein.

Conventional cancer therapies also include targeted therapies for cancer including but not limited to e.g., Ado-trastuzumab emtansine (Kadcyla) targeting HER2 (ERBB2/neu) (approved for use in Breast cancer); Afatinib (Gilotrif) targeting EGFR (HER1/ERBB1), HER2 (ERBB2/neu) (approved for use in Non-small cell lung cancer); Aldesleukin (Proleukin) targeting (approved for use in Renal cell carcinoma, Melanoma); Alectinib (Alecensa) targeting ALK (approved for use in Non-small cell lung cancer); Alemtuzumab (Campath) targeting CD52 (approved for use in B-cell chronic lymphocytic leukemia); Atezolizumab (Tecentriq) targeting PD-L1 (approved for use in Urothelial carcinoma, Non-small cell lung cancer); Avelumab (Bavencio) targeting PD-L1 (approved for use in Merkel cell carcinoma); Axitinib (Inlyta) targeting KIT, PDGFRβ, VEGFR1/2/3 (approved for use in Renal cell carcinoma); Belimumab (Benlysta) targeting BAFF (approved for use in Lupus erythematosus); Belinostat (Beleodaq) targeting HDAC (approved for use in Peripheral T-cell lymphoma); Bevacizumab (Avastin) targeting VEGF ligand (approved for use in Cervical cancer, Colorectal cancer, Fallopian tube cancer, Glioblastoma, Non-small cell lung cancer, Ovarian cancer, Peritoneal cancer, Renal cell carcinoma); Blinatumomab (Blincyto) targeting CD19/CD3 (approved for use in Acute lymphoblastic leukemia (precursor B-cell));

Bortezomib (Velcade) targeting Proteasome (approved for use in Multiple myeloma, Mantle cell lymphoma); Bosutinib (Bosulif) targeting ABL (approved for use in Chronic myelogenous leukemia); Brentuximab vedotin (Adcetris) targeting CD30 (approved for use in Hodgkin lymphoma, Anaplastic large cell lymphoma); Brigatinib (Alunbrig) targeting ALK (approved for use in Non-small cell lung cancer (ALK+)); Cabozantinib (Cabometyx, Cometriq) targeting FLT3, KIT, MET, RET, VEGFR2 (approved for use in Medullary thyroid cancer, Renal cell carcinoma); Carfilzomib (Kyprolis) targeting Proteasome (approved for use in Multiple myeloma); Ceritinib (Zykadia) targeting ALK (approved for use in Non-small cell lung cancer); Cetuximab (Erbitux) targeting EGFR (HER1/ERBB1) (approved for use in Colorectal cancer, Squamous cell cancer of the head and neck); Cobimetinib (Cotellic) targeting MEK (approved for use in Melanoma); Crizotinib (Xalkori) targeting ALK, MET, ROS1 (approved for use in Non-small cell lung cancer); Dabrafenib (Tafinlar) targeting BRAF (approved for use in Melanoma, Non-small cell lung cancer); Daratumumab (Darzalex) targeting CD38 (approved for use in Multiple myeloma); Dasatinib (Sprycel) targeting ABL (approved for use in Chronic myelogenous leukemia, Acute lymphoblastic leukemia); Denosumab (Xgeva) targeting RANKL (approved for use in Giant cell tumor of the bone); Dinutuximab (Unituxin) targeting B4GALNT1 (GD2) (approved for use in Pediatric neuroblastoma); Durvalumab (Imfinzi) targeting PD-L1 (approved for use in Urothelial carcinoma); Elotuzumab (Empliciti) targeting SLAMF7 (CS1/CD319/CRACC) (approved for use in Multiple myeloma); Enasidenib (Idhifa) targeting IDH2 (approved for use in Acute myeloid leukemia); Erlotinib (Tarceva) targeting EGFR (HER1/ERBB1) (approved for use in Non-small cell lung cancer, Pancreatic cancer); Everolimus (Afinitor) targeting mTOR (approved for use in Pancreatic, gastrointestinal, or lung origin neuroendocrine tumor, Renal cell carcinoma, Nonresectable subependymal giant cell astrocytoma, Breast cancer); Gefitinib (Iressa) targeting EGFR (HER1/ERBB1) (approved for use in Non-small cell lung cancer); Ibritumomab tiuxetan (Zevalin) targeting CD20 (approved for use in Non-Hodgkin's lymphoma); Ibrutinib (Imbruvica) targeting BTK (approved for use in Mantle cell lymphoma, Chronic lymphocytic leukemia, Waldenstrom's macroglobulinemia); Idelalisib (Zydelig) targeting PI3Kδ (approved for use in Chronic lymphocytic leukemia, Follicular B-cell non-Hodgkin lymphoma, Small lymphocytic lymphoma); Imatinib (Gleevec) targeting KIT, PDGFR, ABL (approved for use in GI stromal tumor (KIT+), Dermatofibrosarcoma protuberans, Multiple hematologic malignancies); Ipilimumab (Yervoy) targeting CTLA-4 (approved for use in Melanoma); Ixazomib (Ninlaro) targeting Proteasome (approved for use in Multiple Myeloma); Lapatinib (Tykerb) targeting HER2 (ERBB2/neu), EGFR (HER1/ERBB1) (approved for use in Breast cancer (HER2+)); Lenvatinib (Lenvima) targeting VEGFR2 (approved for use in Renal cell carcinoma, Thyroid cancer); Midostaurin (Rydapt) targeting FLT3 (approved for use in acute myeloid leukemia (FLT3+)); Necitumumab (Portrazza) targeting EGFR (HER1/ERBB1) (approved for use in Squamous non-small cell lung cancer); Neratinib (Nerlynx) targeting HER2 (ERBB2/neu) (approved for use in Breast cancer); Nilotinib (Tasigna) targeting ABL (approved for use in Chronic myelogenous leukemia); Niraparib (Zejula) targeting PARP (approved for use in Ovarian cancer, Fallopian tube cancer, Peritoneal cancer); Nivolumab (Opdivo) targeting PD-1 (approved for use in Colorectal cancer, Head and neck squamous cell carcinoma, Hodgkin lymphoma, Melanoma, Non-small cell lung cancer, Renal cell carcinoma, Urothelial carcinoma); Obinutuzumab (Gazyva) targeting CD20 (approved for use in Chronic lymphocytic leukemia, Follicular lymphoma); Ofatumumab (Arzerra, HuMax-CD20) targeting CD20 (approved for use in Chronic lymphocytic leukemia); Olaparib (Lynparza) targeting PARP (approved for use in Ovarian cancer); Olaratumab (Lartruvo) targeting PDGFRα (approved for use in Soft tissue sarcoma); Osimertinib (Tagrisso) targeting EGFR (approved for use in Non-small cell lung cancer); Palbociclib (Ibrance) targeting CDK4, CDK6 (approved for use in Breast cancer); Panitumumab (Vectibix) targeting EGFR (HER1/ERBB1) (approved for use in Colorectal cancer); Panobinostat (Farydak) targeting HDAC (approved for use in Multiple myeloma); Pazopanib (Votrient) targeting VEGFR, PDGFR, KIT (approved for use in Renal cell carcinoma); Pembrolizumab (Keytruda) targeting PD-1 (approved for use in Classical Hodgkin lymphoma, Melanoma, Non-small cell lung cancer (PD-L1+), Head and neck squamous cell carcinoma, Solid tumors (MSI-H)); Pertuzumab (Perjeta) targeting HER2 (ERBB2/neu) (approved for use in Breast cancer (HER2+)); Ponatinib (Iclusig) targeting ABL, FGFR1-3, FLT3, VEGFR2 (approved for use in Chronic myelogenous leukemia, Acute lymphoblastic leukemia); Ramucirumab (Cyramza) targeting VEGFR2 (approved for use in Colorectal cancer, Gastric cancer or Gastroesophageal junction (GEJ) adenocarcinoma, Non-small cell lung cancer); Regorafenib (Stivarga) targeting KIT, PDGFRβ, RAF, RET, VEGFR1/2/3 (approved for use in Colorectal cancer, Gastrointestinal stromal tumors, Hepatocellular carcinoma); Ribociclib (Kisqali) targeting CDK4, CDK6 (approved for use in Breast cancer (HR+, HER2−)); Rituximab (Rituxan, Mabthera) targeting CD20 (approved for use in Non-Hodgkin's lymphoma, Chronic lymphocytic leukemia, Rheumatoid arthritis, Granulomatosis with polyangiitis); Rituximab/hyaluronidase human (Rituxan Hycela) targeting CD20 (approved for use in Chronic lymphocytic leukemia, Diffuse large B-cell lymphoma, Follicular lymphoma); Romidepsin (Istodax) targeting HDAC (approved for use in Cutaneous T-cell lymphoma, Peripheral T-cell lymphoma); Rucaparib (Rubraca) targeting PARP (approved for use in Ovarian cancer); Ruxolitinib (Jakafi) targeting JAK1/2 (approved for use in Myelofibrosis); Siltuximab (Sylvant) targeting IL-6 (approved for use in Multicentric Castleman's disease); Sipuleucel-T (Provenge) targeting (approved for use in Prostate cancer); Sonidegib (Odomzo) targeting Smoothened (approved for use in Basal cell carcinoma); Sorafenib (Nexavar) targeting VEGFR, PDGFR, KIT, RAF (approved for use in Hepatocellular carcinoma, Renal cell carcinoma, Thyroid carcinoma); Temsirolimus (Torisel) targeting mTOR (approved for use in Renal cell carcinoma); Tositumomab (Bexxar) targeting CD20 (approved for use in Non-Hodgkin's lymphoma); Trametinib (Mekinist) targeting MEK (approved for use in Melanoma, Non-small cell lung cancer); Trastuzumab (Herceptin) targeting HER2 (ERBB2/neu) (approved for use in Breast cancer (HER2+), Gastric cancer (HER2+)); Vandetanib (Caprelsa) targeting EGFR (HER1/ERBB1), RET, VEGFR2 (approved for use in Medullary thyroid cancer); Vemurafenib (Zelboraf) targeting BRAF (approved for use in Melanoma); Venetoclax (Venclexta) targeting BCL2 (approved for use in Chronic lymphocytic leukemia); Vismodegib (Erivedge) targeting PTCH, Smoothened (approved for use in Basal cell carcinoma); Vorinostat (Zolinza) targeting HDAC (approved for use in Cutaneous T-cell lymphoma); Ziv-aflibercept (Zaltrap) targeting PIGF, VEGFA/B (approved for use in Colorectal cancer); and the like. These antibodies may be administered as a combination therapy with an anti-ABCC4 antibody provided herein.

Biological response modifiers suitable for use in connection with the methods of the present disclosure include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) interferon-α.; (7) interferon-γ; (8) colony-stimulating factors; (9) inhibitors of angiogenesis; and (10) antagonists of tumor necrosis factor.

Chemotherapeutic agents or antineoplastic agents are non-peptidic (i.e., non-proteinaceous) compounds that reduce proliferation of cancer cells, and encompass cytotoxic agents and cytostatic agents. Non-limiting examples of chemotherapeutic agents include alkylating agents (e.g., nitrosoureas), antimetabolites (e.g., methotrexate), antitumor antibiotics (e.g., anthracyclins), plant alkaloids (e.g., vinca alkaloids, taxanes, etc.), toposiomerase inhibitors, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis). Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, paclitaxel-xylose, or protein bound paclitaxel such as Abraxane®).

Also included within the term “taxane” are a variety of known derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Useful immunotherapies include anti-PD-1/PD-L1 immunotherapies, and/or other immunotherapy targets, such as e.g., immune check point markers, such as CTLA-4, LAG-3 and TIM-3, that may be targeted in treatment methods. Anti-PD-1/PD-L1 immunotherapies which include but are not limited to e.g., those therapies that include administering to a subject an effective amount of one or more anti-PD-1/PD-L1 therapeutic antagonists where such antagonists include but are not limited to e.g., OPDIVO® (nivolumab), KEYTRUDA® (pembrolizumab), Tecentriq™ (atezolizumab), durvalumab (MED14736), avelumab (MSB0010718C), BMS-936559 (MDX-1105), CA-170, BMS-202, BMS-8, BMS-37, BMS-242 and the like. These antibodies may be administered as a combination therapy with an anti-ABCC4 antibody provided herein.

CTLA-4, also known as CD152, binds to CD80 and CD86. Antibodies against CTLA-4 have been approved for treating some cancer types. The co-inhibitory effect of CTLA-4 with other immunotherapies make CTLA-4 a good candidate for use in combination with other immunotherapies to treat certain cancers. TIM-3 may also be targeted for immunotherapy for several cancer types.

LAG-3 is in clinical trials for treating cancers. Anti-LAG-3 immunotherapies include those that employ antagonist LAG-3 antibodies that can both activate T effector cells (by downregulating the LAG-3 inhibiting signal into pre-activated LAG-3+ cells) and inhibit induced (i.e. antigen-specific) Treg suppressive activity. Useful LAG-3 antagonistic antibodies include relatlimab (BMS-986016; developed by Bristol-Myers Squibb), IMP701 (developed by Immutep), TSR-033 (anti-LAG-3 mAb; developed by TESARO, Inc.), and the like.

Immunotherapies also include T cell-based immunotherapies such as e.g., adoptive cell therapy (ACT) and chimeric antigen receptor (CAR) T cell therapies. For example, a subject may be administered a population of CAR T cells engineered to target an antigen expressed by the subject's cancer. A T cell-based therapy may involve, in some instances, obtaining a cellular sample from a subject, such as a blood sample or a tumor biopsy, and culturing immune cells from the sample ex vivo, with or without genetic modification of the cultured immune cells. As an example, immune cells may be obtained from a subject, cultured ex vivo and modified with a CAR specific for an antigen expressed by the cancer to produce a population of CAR T cells. Then, the CAR T cells may be reintroduced into the subject to target the cancer. T cell-based immunotherapies may be configured in various ways, e.g., by targeting various antigens, by collecting/culturing various cell types, etc., depending on a particular cancer to be treated. In addition, T cell-based immunotherapies may be administered systemically, e.g., by intravenous injection, or locally, e.g., by infusion (e.g., intraperitoneal infusion, pleural catheter infusion, etc.), direct injection, and the like.

In some instances, a method of treatment described herein may include administering to a subject one or more inhibitors of a multidrug resistance transporter, including but not limited to e.g., a multidrug resistance transporter other than ABCC4. Useful inhibitors of multidrug resistance transporters include e.g., tyrosine kinase inhibitors, natural products, microRNAs, and small molecule inhibitors. Inhibitors of multidrug resistance transporters include ABC transporter inhibitors.

Individuals suitable for treatment using a method of the present disclosure include an individual having a cancer; an individual diagnosed as having a cancer; an individual being treated for a cancer with chemotherapy, radiation therapy, antibody therapy, surgery, etc.); an individual who has been treated for a cancer (e.g., with one or more of chemotherapy, radiation therapy, antibody therapy, surgery, etc.), and who has failed to respond to the treatment; an individual who has been treated fora cancer (e.g., with one or more of chemotherapy, radiation therapy, antibody therapy, surgery, etc.), and who initially responded to the treatment but who subsequently relapsed, i.e., the cancer recurred.

The methods of the present disclosure may be employed to target and treat a variety of cancers, including e.g., primary cancer, secondary cancers, re-growing cancers, recurrent cancers, refractory cancers and the like. For example, in some instances, the methods of the present disclosure may be employed as an initial treatment of a primary cancer identified in a subject. In some instances, the methods of the present disclosure may be employed as a non-primary (e.g., secondary or later) treatment, e.g., in a subject with a cancer that is refractory to a prior treatment, in a subject with a cancer that is re-growing following a prior treatment, in a subject with a mixed response to a prior treatment (e.g., a positive response to at least one tumor in the subject and a negative or neutral response to at least a second tumor in the subject), and the like.

In some instances, the methods of the present disclosure may be employed to treat a subject with a drug resistant cancer, such as a multi-drug resistant cancer. Multidrug resistance (MDR) is the mechanism by which many cancers develop resistance to chemotherapy drugs, resulting in minimal cell death and the expansion of drug-resistant tumors. MDR cancers may involve one or more resistance mechanisms including but not limited to e.g., increased expression of efflux pumps, decreased absorption of drug, inhibition of cell death or apoptosis, modulating drug metabolism, and the like. In some instances, the methods of the present disclosure may prevent, reverse or circumvent MDR.

In some instances, methods of the present disclosure may include treating a subject having a cancer that is resistant to a first agent with an effective amount of a subject antibody described herein in combination with a second agent that is different from the first agent. For example, in some instances, cancer of a subject may be resistant to a first chemotherapeutic and the subject may be treated by administering an effective amount of a subject antibody as described herein in combination with a second chemotherapeutic that is different from the first. Various combinations of first and second chemotherapeutics may be employed depending on e.g., the type of cancer to be treated, the likelihood of developing resistance, etc.

Numerous cancers are known to develop drug resistance. For this and other reasons the methods of the present disclosure may find use in treating various cancers including but not limited to, e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, ect.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

The methods of treating described herein may, in some instances, be performed in a subject that has previously undergone one or more conventional treatments. For example, in the case of oncology, the methods described herein may, in some instances, be performed following a conventional cancer therapy including but not limited to e.g., conventional chemotherapy, conventional radiation therapy, conventional immunotherapy, surgery, etc. In some instances, the methods described herein may be used when a subject has not responded to or is refractory to a conventional therapy. In some instances, the methods described herein may be used when a subject has responded to a conventional therapy.

In some instances, the method of the present disclosure may be employed to target, treat or clear a subject for minimal residual disease (MRD) remaining after a prior cancer therapy. Targeting, treating and/or clearance of MRD may be pursued using the instant methods whether the MRD is or has been determined to be refractory to the prior treatment or not. In some instances, a method of the present disclosure may be employed to target, treat and/or clear a subject of MRD following a determination that the MRD is refractory to a prior treatment or one or more available treatment options other than those employing the herein described multi-specific antibodies.

In some instances, the instant methods may be employed prophylactically for surveillance. For example, a subject in need thereof may be administered a treatment involving one or more of the herein described antibodies when the subject does not have detectable disease but is at risk of developing a recurrent cancer, including e.g., a drug resistant cancer. In some instances, a prophylactic approach may be employed when a subject is at particularly high risk of developing a primary cancer that would be predicted to be drug resistant or expected to become drug resistant. In some instances, a prophylactic approach may be employed when a subject has been previously treated for a cancer and is at risk of reoccurrence or development of drug resistance.

In some instances, methods of the present disclosure may involve analyzing a cancer for expression of one or more markers or therapeutic targets. For example, in some instances, methods may involve analyzing a sample of a cancer from a subject to determine whether the cancer expresses ABCC4 above a predetermined threshold.

In some instances, whether a subject is treated with an antibody of the present disclosure may depend on the results of ABCC4 expression assessment. For example, in some instances, if a cancer expresses ABCC4 at or above a predetermined threshold then the subject may be treated with an anti-ABCC4 antibody of the present disclosure and if the cancer expresses ABCC4 below the predetermined threshold then the subject may not be treated with an anti-ABCC4 antibody of the present disclosure.

Any convenient assay may be employed for analyzing ABCC4 levels, including but not limited to e.g., flow cytometry, nucleic acid-based assays (e.g., amplification, sequencing, etc.), cell cytometry, immunohistochemistry, and the like. Any convenient biological sample may be employed, including but not limited to e.g., cancer biopsy samples. Useful predetermined thresholds for assessing expression of one or more markers and/or targets may be determined by any convenient and appropriate method, including comparison of the measured level of expression to a corresponding control. For example, in some instances, a useful predetermined threshold for the level of ABCC4 in a sample may correspond to a level of ABCC4 measured in a reference cell, such as a healthy/normal cell.

Methods of Making

As summarized above, methods of the present disclosure also include methods or making and/or identifying antibodies as described herein. A subject antibody can be produced by any known method, e.g., conventional synthetic methods for protein synthesis; recombinant DNA methods; etc.

Where a subject antibody is a single chain polypeptide, it can be synthesized using standard chemical peptide synthesis techniques. Where a polypeptide is chemically synthesized, the synthesis may proceed via liquid-phase or solid-phase. Solid phase polypeptide synthesis (SPPS), in which the C-terminal amino acid of the sequence is attached to an insoluble support followed by sequential addition of the remaining amino acids in the sequence, is an example of a suitable method for the chemical synthesis of a subject antibody. Various forms of SPPS, such as Fmoc and Boc, are available for synthesizing a subject antibody.

Standard recombinant methods can be used for production of a subject antibody. For example, nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. The expression control sequences can be eukaryotic promoter systems in vectors capable of transforming or transfecting eukaryotic host cells (e.g., COS or CHO cells). Once the vector has been incorporated into the appropriate host, the host is maintained under conditions suitable for high level expression of the nucleotide sequences, and the collection and purification of the antibodies.

Because of the degeneracy of the genetic code, a variety of nucleic acid sequences can encode each immunoglobulin amino acid sequence. The desired nucleic acid sequences can be produced by de novo solid-phase DNA synthesis or by polymerase chain reaction (PCR) mutagenesis of an earlier prepared variant of the desired polynucleotide. Oligonucleotide-mediated mutagenesis is an example of a suitable method for preparing substitution, deletion and insertion variants of target polypeptide DNA. See Adelman et al., DNA 2:183 (1983). Briefly, the target polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer, and encodes the selected alteration in the target polypeptide DNA.

Suitable expression vectors are typically replicable in the host organisms either as episomes or as an integral part of the host chromosomal DNA. Commonly, expression vectors contain selection markers (e.g., ampicillin-resistance, hygromycin-resistance, tetracycline resistance, kanamycin resistance or neomycin resistance) to permit detection of those cells transformed with the desired DNA sequences.

Escherichia coli is an example of a prokaryotic host cell that can be used for cloning a subject antibody-encoding polynucleotide. Other microbial hosts suitable for use include bacilli, such as Bacillus subtilis, and other enterobacteriaceae, such as Salmonella, Serratia, and various Pseudomonas species.

Other microbes, such as yeast, are also useful for expression. Saccharomyces (e.g., S. cerevisiae) and Pichia are examples of suitable yeast host cells, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian cells (e.g., mammalian cells grown in in vitro cell culture) can also be used to express and produce the polypeptides of the present invention (e.g., polynucleotides encoding immunoglobulins or fragments thereof). See Winnacker, From Genes to Clones, VCH Publishers, N.Y., N.Y. (1987). Suitable mammalian host cells include CHO cell lines, various Cos cell lines, HeLa cells, HEK cells, myeloma cell lines, and transformed B-cells or hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Examples of suitable expression control sequences are promoters derived from immunoglobulin genes, SV40, adenovirus, bovine papilloma virus, cytomegalovirus and the like. See Co et al., J. Immunol. 148:1149 (1992).

Once synthesized (either chemically or recombinantly), the whole antibodies, their dimers, individual light and heavy chains, or other forms of a subject antibody (e.g., scFv, etc.) can be purified according to standard procedures of the art, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject antibody can be substantially pure, e.g., at least about 80% to 85% pure, at least about 85% to 90% pure, at least about 90% to 95% pure, or 98% to 99%, or more, pure, e.g., free from contaminants such as cell debris, macromolecules other than a subject antibody, etc.

Kits

Aspects of the present disclosure also include kits. The kits may include, e.g., any combination of the antibodies, reagents, compositions, formulations, cells, nucleic acids, expression vectors, or the like, described herein. A subject kit can include one or more of: a subject antibody, a nucleic acid encoding the same, or a cell comprising a subject nucleic acid. Kits may be configured for various purposes, including e.g., treatment kits (e.g., where a kit may include an anti-ABCC4 antibody and e.g., one or more additional active agents, such as a chemotherapeutic), kits for producing antibodies, kits for screening antibodies, and the like.

Optional components of the kit will vary and may, e.g., include: a buffer; a protease inhibitor; etc. Where a subject kit comprises a subject nucleic acid, the nucleic acid may also have restrictions sites, multiple cloning sites, primer sites, etc. The various components of the kit may be present in separate containers or certain compatible components may be pre-combined into a single container, as desired.

In addition to above-mentioned components, a subject kit can include instructions for using the components of the kit to practice a subject method. The instructions for practicing a subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. compact disc-read only memory (CD-ROM), digital versatile disk (DVD), diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1: Generation of antibodies that bind specifically to cells expressing ABCC4

### Materials and Methods

Antibody Generation

Wild type (WT) human and cynolomgus ABCC4, full length and truncated versions, were used to immunize mice or rats. Spleen and lymph node cells from the vaccinated animals were fused with SP2/0 myeloma cells (hybridoma technology). Hybridoma supernatants were screened for the presence of anti-ABCC4 antibodies by flow cytometry. CDRs from selected murine IgGs were cloned into mammalian IgG1 backbone expression vectors for full-length IgG1 antibody expression and production in HEK 293 host cells via transfection using standard protocols and as described below.

### Expression Vectors

For the generation of the antibody expression vectors, the variable regions of heavy and light chain DNA sequences were subcloned in frame with either the human IgG1 constant heavy chain or the human IgG1 kappa constant light chain pre-inserted into the respective generic recipient expression vectors optimized for expression in mammalian cell lines. The genes to be expressed were cloned into the pCl-neo Mammalian Expression Vector (Promega) that uses the full-length human cytomegalovirus (CMV) immediate-early promoter for high level gene expression. The two antibody chains were cloned into two different vectors.

The N-terminal signal sequences from mouse IgG heavy chain and kappa light chain were used for the secreted expression of the heavy and light chain, respectively. The signal peptide was cleaved during expression, leaving intact N-terminus. In the Fab constructs, the C-terminus of the CH1 IgG1 constant region was fused with a 6× His tag for purification.

### Production of mAb

Antibody constructs were expressed using polymer-based co-transfection of Expi293 cells (A14527, ThermoFisher) cells growing in suspension with the mammalian expression vectors following the manufacturer's recommendations.

About six days after transfection the cells were harvested by centrifugation. In detail, 1 ug of total encoding DNA per 1 ml of transfected culture was diluted into of Opti-MEMO medium (Life Technologies), and incubated with Expifectamine reagent (Life Technologies) in the same medium for 20 min. The mixture was then added into the Expi2930 cells growing in suspension in Expi2930 Expression medium (Life Technologies) at 2.5 million cells/ml at 37° C. with and overlay of 8% of CO2 in air. After 6 days, the medium containing the antibody construct was harvested by centrifugation.

Purification of mAbs

To purify antibody formats containing the human Fc, 10 μl of MabSelect™ SuRe™ (GE Healthcare) per 1 ml of supernatant were added to the harvested medium and kept stirring at 4° C. overnight. The next day, the protein A resin was applied in a 24 well filter plate using a vacuum manifold unit (Pall Lifesciences, USA). The resin was washed with PBS and the antibody eluted in 50 mM phosphate pH 3 and neutralized with 10× PBS pH 13.

Analytical Test for mAbs (GXII Reduced and Non-Reduced)

Purity and monomer content of the final protein preparation was determined by high-throughput analysis on the Caliper's LabChip GXII using Protein Express LabChip Kit (Perkin-Elmer) as described by the manufacturer. The chip was automatically primed on the instrument with polymer solution containing 0.2% SDS and fluorescent staining dye. The destain channels were filled with polymer solution free of SDS and dye. Briefly, proteins in reducing and not reducing conditions were prepared by mixing a small volume (2-5 μL) of sample with the caliper sample buffer with or without DDT. The samples were denatured at 75° C. for 5 minutes, centrifuged at 2000 g for 3 minutes, and then run. Electropherograms were generated by LabChip GXII Touch software (Perkin Elmer).

Analytical Test for mAbs (HPLC)

Purity and monomer content of the final protein preparation was determined by high-throughput analysis on HPLC. Size exclusion chromatography (SEC) was performed using an Advancebio SEC 300A 4.6×300 mm, 2.7 um (p/n PL1580-5301) (Agilent Technologies) on an Infinity 1260 Agilent HPLC system. Injections were made under isocratic elution conditions using a mobile phase of PBS, 400 mM sodium chloride, pH 7.4, and detected with absorbance at 280 nm. Quantification is based on the relative area of detected peaks.

A subject antibody can be substantially pure, e.g., at least about 80% to 85% pure, at least about 85% to 90% pure, at least about 90% to 95% pure, or 98% to 99%, or more, pure, e.g., free from contaminants such as cell debris, macromolecules other than a subject antibody, etc.

### Monoclonal Antibody Titration Binding to ABCC4

Binding titration of recombinant antibodies to ABCC4 transfectants was performed by serial dilution of antibodies from about 666 nM. Diluted antibody in flow cytometry buffer was incubated with cells on ice for 30 min. After 2 washes with flow cytometry buffer, bound antibody was detected with PE-labeled F(ab′)₂ fragment goat anti-human IgG (Jackson ImmunoResearch) diluted 1:200 in flow cytometry buffer and incubated with cells for 20 min on ice. After 2 washes with flow cytometry buffer fluorescence was measured on an Attune N×T flow cytometer. Data were analyzed with GraphPad Prism 8.0 software to determine EC50's.

### Stable Cell Lines

Human embryonic kidney HEK 293T cells and murine C6 (ATCC CCL-107) were transfected with human or cynomolgus ABCC4 cDNA with C-terminal fused FLAG tag in a pMONO-hygro vector with Lipofectamine 3000 (Invitrogen #L3000001). The cells were treated with 0.2 mg/ml and 0.5 mg/ml hygromycin respectively for about 2 weeks while cultivated in DMEM and 10% FBS with 0.2 mg/ml hygromycin and 0.5 mg/ml hygromycin, respectively. The cells were washed with PBS, and stained with 10 pg/ml of anti-FLAG-PE after fixing and permeablization. Clones expressing ABCC4 were single-cell sorted into 200 μl DMEM with 10% FBS using a BD Aria FACS machine and cultivated in 96 well plates using standard techniques in an incubator at 37° C. and 5% CO 2. Two weeks later clones expressing high levels of ABCC4 were screened and amplified.

### Cell Binding Assays

Antibody binding to cells was evaluated by flow cytometry. Murine C6 cells stably transfected to express human or cynomolgus ABCC4 were washed once in flow cytometry buffer (PBS+2% FBS+0.02% sodium azide), resuspended at 2×10\hat{ }6 cells/mL in flow cytometry buffer, and dispensed into 96-well microtiter plates at 0.1 mL/well. Recombinant antibodies were added to cells at 5 ug/mL for initial binding confirmation, or serially diluted from 100 ug/mL in flow cytometry buffer. After incubating cells on ice for 30 min, cells were washed twice with flow cytometry buffer. Bound antibody was detected with PE-labeled F(ab′)₂ fragment goat anti-human IgG (Jackson ImmunoResearch) and evaluated on an Attune N×T flow cytometer. EC50 is calculated to be the concentration of antibody that gives half maximal response.

Results

Table 3 lists the following characteristics of the rat anti-ABCC4 antibodies tested: binding to C6 cells stably transfected to express human ABCC4 measured by FACS and binding to C6 cells stably transfected to express cynomolgus ABCC4 measured by FACS. Negative (−) is defined as maximum response (Rₘₐₓ(MFI)) below background level. Positive (+) is defined as MFI>1000.

| | Human | Cynomolgus |
|---|---|---|
| | ABCC4 | ABCC4 |
| Antibody | binding | binding |
| C4.884 | + | − |
| C4.886 | + | − |
| C4.925 | + | − |
| C4.953 | + | − |
| C4.961 | + | + |
| C4.972 | + | − |
| C4.976 | + | + |
| C4.985 | + | − |
| C4.987 | + | − |
| C4.991 | + | − |
| C4.1000 | + | − |
| C4.947 | + | − |

As shown in Table 3, in this study, anti-ABCC4 antibodies C4.961 and C4.976 were found to bind to both human and cynomolgus ABCC4 expressed on cell surface.

In a different experiment, using a different C6.cC4 cell line, detecting the binding of anti-ABCC4 antibodies to C6 cells stably transfected to express human ABCC4 measured by FACS and to C6 cells stably transfected to express cynomolgus ABCC4 measured by FACS, the results set forth in Table 4 were obtained. Negative (−)=Rₘₐₓ (MFI) below background level. Positive (+)=MFI>3× background level.

| | Human | Cynomolgus |
|---|---|---|
| | ABCC4 | ABCC4 |
| Antibody | binding | binding |
| C4.884 | + | − |
| C4.886 | + | + |
| C4.925 | + | − |
| C4.927 | + | − |
| C4.947 | + | − |
| C4.953 | + | + |
| C4.956 | + | − |
| C4.957 | + | + |
| C4.961 | + | + |
| C4.970 | + | − |
| C4.972 | + | + |
| C4.976 | + | + |
| C4.981 | + | − |
| C4.985 | + | − |
| C4.987 | + | − |
| C4.991 | + | − |
| C4.994 | + | − |
| C4.1000 | + | − |
| C4.1006 | + | − |
| C4.1047A | + | − |
| C4.1052 | + | − |
| C4.1054 | + | + |
| C4.1059 | + | − |
| C4.1069 | + | + |
| C4.1071 | + | − |
| C4.1073 | + | + |
| C4.1075 | + | + |
| C4.1078 | + | − |
| C4.1131 | + | + |
| C4.1134 | + | + |
| C4.1138 | + | − |
| C4.1139 | + | − |
| C4.1143 | + | + |
| C4.1145 | + | − |
| C4.1146 | + | − |
| C4.1148 | + | − |
| C4.1152 | + | + |
| C4.1155 | + | − |
| C4.1164 | + | + |

As shown in Table 4, in this binding study anti-ABCC4 antibodies C4.886, C4.953, C4.957, C4.961, C4.972, C4.976, C4.1054, C4.1069, C4.1073, C4.1075, C4.1131, C4.1134, C4.1143, C4.1152, and C4.1164 bind to both human and cynomolgus ABCC4 expressed on cell surface.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. An antibody that specifically binds to ATP Binding Cassette Subfamily C Member 4 (ABCC4) on surface of a mammalian cell, wherein the antibody competes for binding to ABCC4 with an antibody comprising:
heavy chain complementarity determining regions 1-3 (HCDRs 1-3) and light chain CDRs 1-3 (LCDRs 1-3) of a pair of variable heavy chain (VH) region and variable light chain (VL) region of an antibody listed in Table 2.

2. The antibody according to claim 1, wherein the antibody comprises HCDRs 1-3 of the VH region of the antibody listed in Table 2.

3. The antibody according to claim 1 or claim 2, wherein the antibody comprises LCDRs 1-3 of the VL region of the antibody listed in Table 2.

4. The antibody of claim 1, antibody comprising:
heavy chain complementarity determining regions (HCDRs) and light chain CDRs (LCDRs) of a pair of variable heavy chain (VH) region and variable light chain (VL) region of an antibody listed in Table 2.

5. An antibody molecule that specifically binds to ATP Binding Cassette Subfamily C Member 4 (ABCC4) on surface of a mammalian cell and (i) inhibits efflux activity of ABCC4 and/or (ii) binds to both human ABCC4 and cynomolgus ABCC4.

6. The antibody molecule according to claim 5, wherein the antibody comprises:
(a) a variable heavy chain (VH) region comprising heavy chain complementarity determining regions 1-3 (HCDRs 1-3) of a VH region of an antibody listed in Table 2;
(b) a variable light chain (VL) region comprising light chain CDRs 1-3 (LCDRs 1-3) of a VL region of an antibody listed in Table 2;
(c) a VH region comprising HCDRs 1-3 of a VH region of an antibody listed in Table 2 and a VL region comprising LCDRs 1-3 of a VL region of an antibody listed in Table 2; or
(d) a VH region comprising HCDRs 1-3 of a VH region of an antibody listed in Table 2 and a VL region comprising LCDRs 1-3 of a VL region of the antibody.

7. The antibody molecule according to claim 6, wherein the antibody comprises HCDRs 1-3 and LCDRs 1-3 of a pair of VH region and VL region of an antibody listed in Table 2.

8. The antibody according to claim 6, wherein the antibody comprises the HCDRs 1-3 of the VH region of a first antibody listed in Table 2.

9. The antibody molecule according to claim 8, wherein the antibody comprises the LCDRS 1-3 of the VL region of a second antibody in Table 2.

10. The antibody molecule according to claim 6, wherein the antibody molecule comprises the variable light (VL) chain and/or the variable heavy (VH) chain of an antibody listed in Table 2.

11. The antibody molecule according to any of the preceding claims, wherein the antibody, when bound to a cell expressing ABCC4, inhibits efflux by the ABCC4.

12. The antibody molecule according to any of the preceding claims, wherein the antibody comprises a humanized variable light chain.

13. The antibody molecule according to any one of the preceding claims, wherein the antibody comprises a humanized variable heavy chain.

14. The antibody molecule according to any one of the preceding claims, wherein the antibody is selected from the group consisting of a bispecific antibody, an Ig monomer, a Fab fragment, a F(ab′)₂ fragment, a Fd fragment, a scFv, a scAb, a dAb, and a Fv.

15. The antibody molecule according to any one of claims 1-13, wherein the antibody comprises a VL region and a VH region that are present in separate polypeptides.

16. The antibody molecule according to any one of claims 1-13, wherein the antibody comprises a VL region and a VH region that are present in a single polypeptide.

17. The antibody molecule according to any one of claims 1-16 for use in a method of treating cancer in a subject, the method comprising administering the antibody to the subject.

18. The antibody molecule for use according to claim 17, wherein the method comprises administering the antibody in combination with at least one additional active agent wherein the at least one additional active agent comprises a chemotherapeutic agent, an inhibitor of a multidrug resistance transporter, an immunotherapy agent, or a combination thereof.

19. The antibody molecule for use according to claim 18, wherein the at least one additional active agent is a chemotherapeutic agent, optionally wherein the chemotherapeutic agent is a taxol, a vinca alkaloid, an anthracycline, Etoposide, Mitoxantrone, or Methotrexate.

20. The antibody molecule for use according to claim 17-19, wherein the subject being treated has a cancer which has been determined to be resistant to treatment with the chemotherapeutic agent.

21. A pharmaceutical composition comprising:
the antibody of any one of the preceding claims; and
a pharmaceutically acceptable excipient.

22. The pharmaceutical composition according to 21, further comprising an additional active agent.

23. The pharmaceutical composition according to claim 22, wherein the additional active agent is a chemotherapeutic agent.

24. The pharmaceutical composition according to claim 22, wherein the additional active agent comprises an inhibitor of a multidrug resistance transporter.

25. The pharmaceutical composition according to claim 22, wherein the additional active agent comprises an immunotherapy agent.

26. One or more nucleic acids comprising one or more sequences encoding the antibody molecule according to any of claims 1 to 16.

27. One or more recombinant expression vectors comprising the one or more nucleic acids according to claim 26.

28. A host cell genetically modified with the recombinant one or more recombinant expression vectors according to claim 27.

29. An immune effector cell comprising a chimeric antigen receptor (CAR) comprising an ABCC4 binding domain, a transmembrane domain, and an intracellular signaling domain, and wherein the ABCC4 binding domain comprises heavy chain complementarity determining regions 1-3 (HCDRs1-3) of a variable heavy chain (VH) region of an antibody listed in Table 2 and/or light chain CDRs 1-3 (LCDRs 1-3) of a variable light chain (VL) region of an antibody listed in Table 2.

30. A method of assaying expression of ABCC4 on cell surface of a cell, the method comprising contacting the cell with the antibody according to any of claims 1 to 16.

31. The method of claim 30, wherein the antibody is detectably labeled.

32. A method of inhibiting efflux activity of ABCC4 expressed by a live cell, the method comprising contacting the cell with the antibody according to any of claims 1 to 16.

33. The method of claim 32, further comprising contacting the cell with an inhibitor of ABCC4 mediated efflux.

34. The method according to claim 32 or 33, further comprising contacting the cells with a chemotherapy agent.

35. The method according to any one of claims 30 to 34 wherein the cell is a cancer cell.

36. The method according to claim 35, wherein the cancer cell is a multidrug resistant cancer cell.

37. A method of treating cancer comprising administering to a subject an antibody of any one of claims 1, 7, 12, and 13, or a pharmaceutical composition of claim 21.

38. The method of claim 37, wherein the subject is a human patient.

39. The method of claim 38, wherein the antibody is humanized or human.

40. Use of an antibody of antibody of any one of claims 1, 7, 12, and 13, or a pharmaceutical composition of claim 21 in the treatment of a subject with cancer.

41. The use of claim 40, wherein the subject is a human cancer patient.

42. Use of an antibody of any one of claims 1, 7, 12, and 13 in the preparation of a medicament to treat cancer.


SEQUENCE LISTING (241 sequences)

SEQ ID NO: 1 - protein, 113 aa, Artificial sequence
  synthetic sequence
EVQLVESGPGLVQPSQTLSLTCTVSGFSLTSNSVHWVRQPPGKGLEWIGAIWSGGSTDYNSALKSRLSISRDTSKSQVFLKMNSLQTEDTAIYFCTGLIFPYWGQGASVTVSS

SEQ ID NO: 2 - protein, 121 aa, Artificial sequence
  synthetic sequence
EVKLLESGGDLLQPGRSLKLSCAASGFTFNDYNMAWVRQAPKKGLQWVATILYDGSRTYYRDSVKGRFTISRDNSKSTLYLQMDSLRSEDTATYYCTTEFGYGSSHWFACWGQGTLVTVSS

SEQ ID NO: 3 - protein, 119 aa, Artificial sequence
  synthetic sequence
EVQLVESGGGLVQPGRSLKLSCAASGFTFSDYYMAWVRQAPTKGLEWVAYINYDGGSTHYGDSVKGRFTISRDNEKSTLYLQMNSLRSEDMATYYCARENYGYNSFDYWGQGASVTVSS

SEQ ID NO: 4 - protein, 116 aa, Artificial sequence
  synthetic sequence
EVQLVESGPGLVQPSRTLSLTCTVSGFSLTSYGVSWVRQPPGKGLEWIAAIWSGGSTDYNSALKSRLSISRDTSKSQVLLKMNSLQTEDTAMYFCARGATEGIDYWGQGASVTVSS

SEQ ID NO: 5 - protein, 118 aa, Artificial sequence
  synthetic sequence
EVKLVESGGGLVQPGGSMRLSCAASGFTFTDFYMNWIRQPAGKAPEWLGFIRNKANAYTTAYNPSVKGRFTISRDNAQNMLYLQMNTLRAEDTATYYCARSNYGFDYWGQGVMVTVSS

SEQ ID NO: 6 - protein, 120 aa, Artificial sequence
  synthetic sequence
QVTLKESGPGILQPSQTLSLTCTFSGFSLSTYGMGVGWIRQPSGKALEWLANIWWDDDKSYNPSLKNRLTISKGTSNNQAFLKITSVDTADSATYHCARIGSGNSEFDYWGQGVMVTVSS

SEQ ID NO: 7 - protein, 116 aa, Artificial sequence
  synthetic sequence
QLKESGPGLVKPSQSLSLTCSVTGYTITSGYDWTWLRKFPGNKMEWMGYISYSGFTNYNPSLRSRISITRDTSKNQFFLQLNSVTAEDTATYFCTRTRGYNPFPYWGQGTLVTVSS

SEQ ID NO: 8 - protein, 118 aa, Artificial sequence
  synthetic sequence
EVQLKESGPGLVQPSQTLSLTCTVSGFSLTNYGVSWVRQPPGKGLEWIGAIWSGGSTGYNSALKSRLSISRDTSKSQVLLKMNSLQTEDTAMYFCARSGVEGPPFDYWGQGASVTVSS

SEQ ID NO: 9 - protein, 115 aa, Artificial sequence
  synthetic sequence
EVQLKESGPGLVQPSQTLSLTCAVSGFSLTNYHVHWVRQPPGKGLEWMGVMWSDGDTSYNSALKSRLSISRDTSKSQFFLKMNSLQTEDTATYYCARGGVYFDYWGQGASVTVSS

SEQ ID NO: 10 - protein, 121 aa, Artificial sequence
  synthetic sequence
EVQLVESGPGLVQPSQTLSLTCTVAGFSLTSYNVHWVRQPPGKGLEWMGVIWNTGDTRYDSALKSRLSISKDTSKSQVFLNMNSLQTEDTATYYCARDDNVYYGMHAMDAWGQGASVTVSS

SEQ ID NO: 11 - protein, 124 aa, Artificial sequence
  synthetic sequence
EVQLKESGPGLVQPSETLSLTCTVSGFSLTSYNVHWVRQPPGKGLEWMGVMWSGGSTDYSSALKSRLSISRDTSKNQVFLKMNSLQSEDTTTYYCARDRGGYYYDGYPHDFVYWGQGVMVTVSS

SEQ ID NO: 12 - protein, 118 aa, Artificial sequence
  synthetic sequence
EVKLLESGGGLVQPGRSLKVSCVASGFIFSDYYMAWVRQAPTKGLEWVAYINYDGGSTYYGDSVKGRFTISRDNAKSSLYLQMNSLRFEDMATYYCARRDYGSKFDYWGQGVMVTVSS

SEQ ID NO: 13 - protein, 41 aa, Artificial sequence
  synthetic sequence
EVQLVESGGGLVQPKESLKISCAASGFTFSNAAMYWVRQAP

SEQ ID NO: 14 - protein, 121 aa, Artificial sequence
  synthetic sequence
EVQLVESGGGLVQPGRSLKLSCTASGFTFNKNWMAWIRQAPGKGLEWVASITNTGGNTYYPDSVKGRFTVSRDQTISTLYLQMNSLRSEDTATYFCTRERIGSGYYVMDAWGRGASVTVSS

SEQ ID NO: 15 - protein, 120 aa, Artificial sequence
  synthetic sequence
EVQLVESGGGLVQPGRSMKLSCAASGFTFSNYYMAWVRQAPTKGLEWVASISTGGGNTHYRDSVKGRFTISRDNAKSTLYLQMDSLRSEETATYYCARGEDRYKGVFAYWGQGTLVTVSS

SEQ ID NO: 16 - protein, 118 aa, Artificial sequence
  synthetic sequence
EVKLVESGGGLVQPGRSLQVSCVASGFIFTDYYMAWVRQAPTKGLEWVAYINYDGGSTYYGDSVKGRFTISRDNAKSTLYLQMNSLRSEDMATYYCARRDYGSKFDNWGQGTLVTVSS

SEQ ID NO: 17 - protein, 117 aa, Artificial sequence
  synthetic sequence
QIQLVQSGPELKKPGESVKISCKASGYTFTDYAIHWVTQAPGKGLKWMGWISTYTGKTTYTDDFKGRFVFSLDASASTANLQINNLKNEDTAAYFCARGGLQWFDYWGQGTLVTVSS

SEQ ID NO: 18 - protein, 114 aa, Artificial sequence
  synthetic sequence
QVQLTESGPGLVQPSQTLSLTCTISGFSLTSYGVSWVRQPPGKGLEWIGAIWSGGGTNYDSALKSRLSISRDTSKSQVLLKMNSLQTEDTAMYFCASGDWFAYWGQGTLVTVSS

SEQ ID NO: 19 - protein, 115 aa, Artificial sequence
  synthetic sequence
EVQLTESGPGLVQPSQTLSLTCTVSGFSLTSYDVHWVRQPPGKGLEWMGRMQNGGSTDYNSALKSRLSISRDTSKSQVFLKMNSVQTEDTAMYFCVRSFIIRGYWGQGASVTVSS

SEQ ID NO: 20 - protein, 119 aa, Artificial sequence
  synthetic sequence
EVQLMESGPGLVQPSQTLSLTCTVSGLSLISNNINWVRQPPGKGLEWIGAISGGGSAFYNSALKSRLSFSRDTSKSQVFLKMNSLQTEDTAIYFCARGGSEDPDYFDYWGQGVMVTVSS

SEQ ID NO: 21 - protein, 120 aa, Artificial sequence
  synthetic sequence
QVQLLQSGAALVKPGASVKMSCKPSGYTFTDYWVSWVKQSHGKSLEWIGEIYPNNGATNFNEKFKGKATLTVDKSTSTAYMELSRLTSEDSAIYYCPSRKYGYNWNFDYWGQGTMVTVSS

SEQ ID NO: 22 - protein, 110 aa, Artificial sequence
  synthetic sequence
GLVQPSQTLSLTCTVSGFSLNSNGVSWVRQPPGKGLEWIVEISSGGSAFYNSALKSRLSISRDTSKSQVFLKMNSLQTEDTAIYFCTRGGTEDPDYFDYWGQGASVTVSS

SEQ ID NO: 23 - protein, 114 aa, Artificial sequence
  synthetic sequence
EVQWKESGPGLVQPSQTLSLTCTVSGFSLTSYGVSWVRQPPGKGLEWIGAIWSGGGTDYNSALKSRLSISRDTSKSQVLLKMNSLQTEDTAMYFCAGNHYFDYWGQGTMVTVSS

SEQ ID NO: 24 - protein, 126 aa, Artificial sequence
  synthetic sequence
EVQMKESGPGLVQPSQTLSLTCTVSGFSLTSYHVHWVRQPPGKGLEWMGVMWSDGDTSYNSALKSRLSINRDTSQSRVFLKMNSLQTEDTSTYYCARDRDVLYYDGDFSPFVMDAWGQGASVTVSS

SEQ ID NO: 25 - protein, 119 aa, Artificial sequence
  synthetic sequence
EVQLKESGPGLVQPSQTLSLTCTVSGLSLISSNINWVRQPPGKGLEWIGAISSGGSAFYNSALKSRLTSSRDTSKSQVFLKMNSLQTEDTAIYFCARGGSEDPDYFDYWGQGVMVTVSS

SEQ ID NO: 26 - protein, 118 aa, Artificial sequence
  synthetic sequence
EVQLVESGGGLVQPGGSLRLSCAASGFTFTDFYMSWIRQPPGKTPEWLGVIRNKAYVYTTEYNPSVKGRFTISRDNTQNILYLQMNTLRVEDTAIYYCARNWEGFASWGQGTLVTVSS

SEQ ID NO: 27 - protein, 120 aa, Artificial sequence
  synthetic sequence
EVKLVESDGGLVQPRKSLKLSCAASGFTFTDYYMAWVRQGPTMGLEWVATISNDGSTKYYRDSVKGRFTLSRDNAKTTLHLQMDSLRSEDTATYYCARLTIRYKGVMDAWGQGASVTVSS

SEQ ID NO: 28 - protein, 120 aa, Artificial sequence
  synthetic sequence
EVQLVESDGGLVQPGRSLKLSCAASGFTFSDFYMAWVRQAPTKGLEWIATISHDGRNTFYRDSVKGRFTISRDNAKRALYLQMDSLRSEDTATYYCASLTTRYKGVMEAWGQGASVTVSS

SEQ ID NO: 29 - protein, 116 aa, Artificial sequence
  synthetic sequence
EVQLKESGPGLVKPSETLSLTCTVSGFSLTSYHVSWVRQPPGKGLEWMGVIWGDGNTAYNSALKSRLSINRDTSKSQVFLKMNSLQTEDTATYYCARHPYYSIPYWGQGASVTVSS

SEQ ID NO: 30 - protein, 120 aa, Artificial sequence
  synthetic sequence
EVQLVESDGGLVQPGRSLKLSCAASGFTFSDYYMAWVRQAPTKGLYWVASISYDGSTTYYRDSVKGRFTISRDNAKSTLYLQMGSLRSEDTATYYCARLSIRYKGVMDAWGRGASVTVSS

SEQ ID NO: 31 - protein, 120 aa, Artificial sequence
  synthetic sequence
EVQLVESDGGLLQPGRSLKLSCAASGFTFSDFYMAWVRQAPTKGLEWIATISHDGRNTYYRDSVKGRFTISRDNAKRTLYLEMDSLRSEDTATYYCATLTTRYKGVMEAWGQGASVTVSS

SEQ ID NO: 32 - protein, 120 aa, Artificial sequence
  synthetic sequence
EVKLVESGGGLVQPGRSLKLSCAASGFTFSNYDMAWVRQAPTKGLEWVASIIPSGGTSYYRDSVKGRFTVSRDNAKSTLYLQMDSLRSEDTATYYCARLTTSHYYVMDAWGQGASVTVSS

SEQ ID NO: 33 - protein, 120 aa, Artificial sequence
  synthetic sequence
EVQLVESDGGLVQPGRSLKLSCAASGFTFSDYYMAWVRQAPPKGLEWVATISYDAFTTYYRDSVKGRFTISRDNAKTILFLQMDSLRSEDTATYYCIRLSTRYKGVMDAWGHGASVTVSS

SEQ ID NO: 34 - protein, 120 aa, Artificial sequence
  synthetic sequence
EVKLVESDGGLVQPGRSLKLSCAASGFTFSDFYMAWVRQAPTKGLEWIASISHDGRNTYYRDSVKGRFTISRDNAKRTLYLQMDSLRSEDTATYYCASLTARYKGVMGAWGQGTLVTVSS

SEQ ID NO: 35 - protein, 118 aa, Artificial sequence
  synthetic sequence
EVKLLESGGGLVQPGGSLRLSCATSGFSFTDFYMSWIRQPLGKAPEWLGVIRNRAIGFRTEYNPSVKGRFTISRDNTQNILYLQMNTLRTEDTAIYYCARNNLGFDYWGQGASVTVSS

SEQ ID NO: 36 - protein, 119 aa, Artificial sequence
  synthetic sequence
EVQLVESDGGLVQPGRSLKLSCAASGFTFSDYYMAWVRQAPTKGLYWVASISYDGSTTYYRDSVKGRFTISRDNAKSTLYLQMGSLRSEDTATYYCARLSIRYKGVMDAWGRGASVTVS

SEQ ID NO: 37 - protein, 5 aa, Artificial sequence
  synthetic sequence
SNSVH

SEQ ID NO: 38 - protein, 5 aa, Artificial sequence
  synthetic sequence
DYNMA

SEQ ID NO: 39 - protein, 5 aa, Artificial sequence
  synthetic sequence
DYYMA

SEQ ID NO: 40 - protein, 5 aa, Artificial sequence
  synthetic sequence
SYGVS

SEQ ID NO: 41 - protein, 5 aa, Artificial sequence
  synthetic sequence
DFYMN

SEQ ID NO: 42 - protein, 7 aa, Artificial sequence
  synthetic sequence
TYGMGVG

SEQ ID NO: 43 - protein, 16 aa, Artificial sequence
  synthetic sequence
YISYSGFTNYNPSLRS

SEQ ID NO: 44 - protein, 5 aa, Artificial sequence
  synthetic sequence
NYGVS

SEQ ID NO: 45 - protein, 5 aa, Artificial sequence
  synthetic sequence
NYHVH

SEQ ID NO: 46 - protein, 5 aa, Artificial sequence
  synthetic sequence
SYNVH

SEQ ID NO: 47 - protein, 5 aa, Artificial sequence
  synthetic sequence
NAAMY

SEQ ID NO: 48 - protein, 5 aa, Artificial sequence
  synthetic sequence
KNWMA

SEQ ID NO: 49 - protein, 5 aa, Artificial sequence
  synthetic sequence
NYYMA

SEQ ID NO: 50 - protein, 5 aa, Artificial sequence
  synthetic sequence
DYAIH

SEQ ID NO: 51 - protein, 5 aa, Artificial sequence
  synthetic sequence
SYDVH

SEQ ID NO: 52 - protein, 5 aa, Artificial sequence
  synthetic sequence
SNNIN

SEQ ID NO: 53 - protein, 5 aa, Artificial sequence
  synthetic sequence
DYWVS

SEQ ID NO: 54 - protein, 5 aa, Artificial sequence
  synthetic sequence
SNGVS

SEQ ID NO: 55 - protein, 5 aa, Artificial sequence
  synthetic sequence
SYHVH

SEQ ID NO: 56 - protein, 5 aa, Artificial sequence
  synthetic sequence
SSNIN

SEQ ID NO: 57 - protein, 5 aa, Artificial sequence
  synthetic sequence
DFYMS

SEQ ID NO: 58 - protein, 5 aa, Artificial sequence
  synthetic sequence
DFYMA

SEQ ID NO: 59 - protein, 5 aa, Artificial sequence
  synthetic sequence
SYHVS

SEQ ID NO: 60 - protein, 5 aa, Artificial sequence
  synthetic sequence
NYDMA

SEQ ID NO: 61 - protein, 16 aa, Artificial sequence
  synthetic sequence
AIWSGGSTDYNSALKS

SEQ ID NO: 62 - protein, 17 aa, Artificial sequence
  synthetic sequence
TILYDGSRTYYRDSVKG

SEQ ID NO: 63 - protein, 17 aa, Artificial sequence
  synthetic sequence
YINYDGGSTHYGDSVKG

SEQ ID NO: 64 - protein, 19 aa, Artificial sequence
  synthetic sequence
FIRNKANAYTTAYNPSVKG

SEQ ID NO: 65 - protein, 16 aa, Artificial sequence
  synthetic sequence
NIWWDDDKSYNPSLKN

SEQ ID NO: 66 - protein, 16 aa, Artificial sequence
  synthetic sequence
AIWSGGSTGYNSALKS

SEQ ID NO: 67 - protein, 16 aa, Artificial sequence
  synthetic sequence
VMWSDGDTSYNSALKS

SEQ ID NO: 68 - protein, 16 aa, Artificial sequence
  synthetic sequence
VIWNTGDTRYDSALKS

SEQ ID NO: 69 - protein, 16 aa, Artificial sequence
  synthetic sequence
VMWSGGSTDYSSALKS

SEQ ID NO: 70 - protein, 17 aa, Artificial sequence
  synthetic sequence
YINYDGGSTYYGDSVKG

SEQ ID NO: 71 - protein, 19 aa, Artificial sequence
  synthetic sequence
RIRTKPNNYATYYADSVKG

SEQ ID NO: 72 - protein, 17 aa, Artificial sequence
  synthetic sequence
SITNTGGNTYYPDSVKG

SEQ ID NO: 73 - protein, 17 aa, Artificial sequence
  synthetic sequence
SISTGGGNTHYRDSVKG

SEQ ID NO: 74 - protein, 17 aa, Artificial sequence
  synthetic sequence
WISTYTGKTTYTDDFKG

SEQ ID NO: 75 - protein, 16 aa, Artificial sequence
  synthetic sequence
AIWSGGGTNYDSALKS

SEQ ID NO: 76 - protein, 16 aa, Artificial sequence
  synthetic sequence
RMQNGGSTDYNSALKS

SEQ ID NO: 77 - protein, 16 aa, Artificial sequence
  synthetic sequence
AISGGGSAFYNSALKS

SEQ ID NO: 78 - protein, 17 aa, Artificial sequence
  synthetic sequence
EIYPNNGATNFNEKFKG

SEQ ID NO: 79 - protein, 16 aa, Artificial sequence
  synthetic sequence
EISSGGSAFYNSALKS

SEQ ID NO: 80 - protein, 16 aa, Artificial sequence
  synthetic sequence
AIWSGGGTDYNSALKS

SEQ ID NO: 81 - protein, 16 aa, Artificial sequence
  synthetic sequence
AISSGGSAFYNSALKS

SEQ ID NO: 82 - protein, 19 aa, Artificial sequence
  synthetic sequence
VIRNKAYVYTTEYNPSVKG

SEQ ID NO: 83 - protein, 17 aa, Artificial sequence
  synthetic sequence
TISNDGSTKYYRDSVKG

SEQ ID NO: 84 - protein, 17 aa, Artificial sequence
  synthetic sequence
TISHDGRNTFYRDSVKG

SEQ ID NO: 85 - protein, 16 aa, Artificial sequence
  synthetic sequence
VIWGDGNTAYNSALKS

SEQ ID NO: 86 - protein, 17 aa, Artificial sequence
  synthetic sequence
SISYDGSTTYYRDSVKG

SEQ ID NO: 87 - protein, 17 aa, Artificial sequence
  synthetic sequence
TISHDGRNTYYRDSVKG

SEQ ID NO: 88 - protein, 17 aa, Artificial sequence
  synthetic sequence
SIIPSGGTSYYRDSVKG

SEQ ID NO: 89 - protein, 17 aa, Artificial sequence
  synthetic sequence
TISYDAFTTYYRDSVKG

SEQ ID NO: 90 - protein, 17 aa, Artificial sequence
  synthetic sequence
SISHDGRNTYYRDSVKG

SEQ ID NO: 91 - protein, 19 aa, Artificial sequence
  synthetic sequence
VIRNRAIGFRTEYNPSVKG

SEQ ID NO: 92 - protein, 5 aa, Artificial sequence
  synthetic sequence
LIFPY

SEQ ID NO: 93 - protein, 12 aa, Artificial sequence
  synthetic sequence
EFGYGSSHWFAC

SEQ ID NO: 94 - protein, 10 aa, Artificial sequence
  synthetic sequence
ENYGYNSFDY

SEQ ID NO: 95 - protein, 8 aa, Artificial sequence
  synthetic sequence
GATEGIDY

SEQ ID NO: 96 - protein, 7 aa, Artificial sequence
  synthetic sequence
SNYGFDY

SEQ ID NO: 97 - protein, 10 aa, Artificial sequence
  synthetic sequence
IGSGNSEFDY

SEQ ID NO: 98 - protein, 9 aa, Artificial sequence
  synthetic sequence
TRGYNPFPY

SEQ ID NO: 99 - protein, 10 aa, Artificial sequence
  synthetic sequence
SGVEGPPFDY

SEQ ID NO: 100 - protein, 7 aa, Artificial sequence
  synthetic sequence
GGVYFDY

SEQ ID NO: 101 - protein, 16 aa, Artificial sequence
  synthetic sequence
DRGGYYYDGYPHDFVY

SEQ ID NO: 102 - protein, 9 aa, Artificial sequence
  synthetic sequence
RDYGSKFDY

SEQ ID NO: 103 - protein, 8 aa, Artificial sequence
  synthetic sequence
YNSGFLDC

SEQ ID NO: 104 - protein, 12 aa, Artificial sequence
  synthetic sequence
ERIGSGYYVMDA

SEQ ID NO: 105 - protein, 11 aa, Artificial sequence
  synthetic sequence
GEDRYKGVFAY

SEQ ID NO: 106 - protein, 9 aa, Artificial sequence
  synthetic sequence
RDYGSKFDN

SEQ ID NO: 107 - protein, 8 aa, Artificial sequence
  synthetic sequence
GGLQWFDY

SEQ ID NO: 108 - protein, 6 aa, Artificial sequence
  synthetic sequence
GDWFAY

SEQ ID NO: 109 - protein, 7 aa, Artificial sequence
  synthetic sequence
SFIIRGY

SEQ ID NO: 110 - protein, 11 aa, Artificial sequence
  synthetic sequence
GGSEDPDYFDY

SEQ ID NO: 111 - protein, 11 aa, Artificial sequence
  synthetic sequence
RKYGYNWNFDY

SEQ ID NO: 112 - protein, 11 aa, Artificial sequence
  synthetic sequence
GGTEDPDYFDY

SEQ ID NO: 113 - protein, 6 aa, Artificial sequence
  synthetic sequence
NHYFDY

SEQ ID NO: 114 - protein, 18 aa, Artificial sequence
  synthetic sequence
DRDVLYYDGDFSPFVMDA

SEQ ID NO: 115 - protein, 7 aa, Artificial sequence
  synthetic sequence
NWEGFAS

SEQ ID NO: 116 - protein, 11 aa, Artificial sequence
  synthetic sequence
LTIRYKGVMDA

SEQ ID NO: 117 - protein, 11 aa, Artificial sequence
  synthetic sequence
LTTRYKGVMEA

SEQ ID NO: 118 - protein, 8 aa, Artificial sequence
  synthetic sequence
HPYYSIPY

SEQ ID NO: 119 - protein, 11 aa, Artificial sequence
  synthetic sequence
LSIRYKGVMDA

SEQ ID NO: 120 - protein, 11 aa, Artificial sequence
  synthetic sequence
LTTSHYYVMDA

SEQ ID NO: 121 - protein, 11 aa, Artificial sequence
  synthetic sequence
LSTRYKGVMDA

SEQ ID NO: 122 - protein, 11 aa, Artificial sequence
  synthetic sequence
LTARYKGVMGA

SEQ ID NO: 123 - protein, 7 aa, Artificial sequence
  synthetic sequence
NNLGFDY

SEQ ID NO: 124 - protein, 112 aa, Artificial sequence
  synthetic sequence
DVVMTQTPSILSGTLGRSVSISCRSSQSLLDSDGTTYLYWFLQRPGQSPQRLIYLVSNLGSGVPNRFSGSGSGTDFTLKISGVEAEDLGVYYCMQATHVPFTFGSGTKLEIK

SEQ ID NO: 125 - protein, 112 aa, Artificial sequence
  synthetic sequence
DVVLTQTPVSLPVSLGGQASISCRSSQSLVHSNGNTYLHWYLQKPGQSPQLLISRISNRFSGVPDRFSGSGSGTDFTLKISRVEPEDLGDYYCFQSTHFPHTFGAGTKLEIK

SEQ ID NO: 126 - protein, 107 aa, Artificial sequence
  synthetic sequence
DIQVTQSPASLSASLGETISIECLTSEDISSYLTWYQQKPGKSPHLLIYGANSLQAGVPSRFSGSGSGTQYSLKISSMQPEDEGDYFCQQSYKFPLTFGSGTKLELK

SEQ ID NO: 127 - protein, 106 aa, Artificial sequence
  synthetic sequence
AIQVTQSPTSLSASLGDRVTLTCRASQDINNKMAWYQQKPGEVPQLLIYYASTLQSGTPSRFSGSGSGTDFSFTISHLQSEDFATYYCLQGYSLYTFGAGTKLELK

SEQ ID NO: 128 - protein, 112 aa, Artificial sequence
  synthetic sequence
DVAMTQTPPSLSVAIGQSVSISCKSSQSLVYSDGKTYLHWLLQSSGRSPKRLIYQVSNLDSGVPDRFSGTGSQKDFTLKISRVEAKDLGVYYCAQTTHFPFTFGSGTKLEIK

SEQ ID NO: 129 - protein, 107 aa, Artificial sequence
  synthetic sequence
DVVLTQSPSSLSASLGDRVTITCRASQDIGNYLTWFQQKPGKSPRRMIYGATNLAAGVPSRFSGSRSGSDYSLTISSLESEDVADYHCLQSIQYPFTFGSGTKLEIK

SEQ ID NO: 130 - protein, 113 aa, Artificial sequence
  synthetic sequence
DIVMTQSPSSLAVSAGETVTINCKSSQSLLYSGNQKNSLAWYQQKPGQSPKLLIYWASTRQSGVPDRFIGSGSGTDFTLTFSSVQAEDLAIYYCQHYFDTPYTFGAGTKLELK

SEQ ID NO: 131 - protein, 106 aa, Artificial sequence
  synthetic sequence
DIKMIQSPSSLSASLGERVTISCRASENINNYLSWYQKKEDGSVKLLIYHTSRLQPGAPSRFSGSGSGKDYSLTISGLDSEDIATYYCQQGYTPPTFGAGTKLELK

SEQ ID NO: 132 - protein, 110 aa, Artificial sequence
  synthetic sequence
EIVIIQSPALAVSLGQRATISCKTNQNVDYYGNSYIHWYQQKPGQQPKLLIYLASNLASGIPARFSGRGSGTDFTLTIDPVEADDPATYYCQQSRNLPFTFGSGTKLELK

SEQ ID NO: 133 - protein, 112 aa, Artificial sequence
  synthetic sequence
DVVLTQTPSILSATIGQSVSISCRSSQSLLDSDGNTYLYWFLQRPGQSPQRLIYLVSNLGSGVPNRFSGSGSGTDFTLKISGVEAEDLGVFYCMQATHAPFTFGSGTKLEVK

SEQ ID NO: 134 - protein, 106 aa, Artificial sequence
  synthetic sequence
DTVLTQSPALTVSPGERVTISCRASESVTSFMNWYQQKPGQQPKLLIYLASDLESGVPARFSGSGSGADFTLTIDPVEADDTATYYCQQSWIDPPTFGTGTKLELK

SEQ ID NO: 135 - protein, 107 aa, Artificial sequence
  synthetic sequence
DVQMTQSPSNLAASPGESVSINCKASKSISKYLAWYQQKPGKANKLLIYSGSTLQSGTPSRFSGSGSGTDFTLTIRNLEPEDFGLYYCQHHNEYPPTFGAGTKLELK

SEQ ID NO: 136 - protein, 106 aa, Artificial sequence
  synthetic sequence
DIVMTQSPSLLSASVGDRVTLTCKGSQNINNFLAWYQQKRGEAPKLLIYKTNSLHTGIPSRFSGSGSGTEYTLTISSLHSEDLATYYCYQYNNGYTFGAGTKLELK

SEQ ID NO: 137 - protein, 106 aa, Artificial sequence
  synthetic sequence
EILLTQSPTAMAASPGEKVTITCRASFRVSYMHWYQQKPGASPKPWIYDTSKLASGVPTRFSGSGSGTSYSFTISSMETEDTATYYCQQWTSNSRTFGGGTKLVLK

SEQ ID NO: 138 - protein, 106 aa, Artificial sequence
  synthetic sequence
DIQMTQSPPSLSASLGDKVTITCQASQNINKYIAWFQQKPGKAPRLLIRYTSTLESGTPSRFSGSGSGRDYSFSISTVESEDIASYYCLQYVNLYTFGAGTKLELK

SEQ ID NO: 139 - protein, 112 aa, Artificial sequence
  synthetic sequence
DIVMTQGALPNPVPSGESASITCQSSKSLLYSDGKTYLNWYLQRPGQSPQLLIYWMSIRASGVSDRFSGSGSGTDFTLKISRVEAEDVGVYYCQQFLEFPYTFGAGTKLEIK

SEQ ID NO: 140 - protein, 113 aa, Artificial sequence
  synthetic sequence
ETVMTQTPSSLAVSAGETVTINCRSSQSLLYSGNQKNYLAWYQQKPGQSPKLLIYWSSTRQYGVPDRFIGSGSGTDFTLTISSVQAEDLAIYYCQQYYDTPLTFGSGTKLELK

SEQ ID NO: 141 - protein, 113 aa, Artificial sequence
  synthetic sequence
DIVMTQSPSSLAVSAGETVTINCKSSQSLLYSGNQKNYLAWYQQKPGQSPKLLIYWSSTRQSGVPDRFMGSGSGTDFTLTISSVQAEDLAIYYCQQYYDTPLTFGSGTKLEIK

SEQ ID NO: 142 - protein, 110 aa, Artificial sequence
  synthetic sequence
DIVLTQSPALAVSLGQRATISCRARQSVSISRYNLMHWYQQKPGQQPKVLIYRASNLASGIPARFSGSGSGTDFTLTIDPVQADDIATYYCQQSRDSPWTFGGGTKLELK

SEQ ID NO: 143 - protein, 110 aa, Artificial sequence
  synthetic sequence
DIVLTQSPALAVSLGQRATISCWASQSVSISRYNLMHWYQQKPGQQPKLLIYRASNLASGIPARFSGSGSGTDFTLTINPVQADDIATYYCQQSGESPWTFGGGTKLELK

SEQ ID NO: 144 - protein, 113 aa, Artificial sequence
  synthetic sequence
DIVLTQSPSSLAVSAGETVTINCKSSQSLLYSGNQKNYLAWYQQTPGQSPKLLIYWASTRQSGVPDRFIGSGSGTDFILTITSVQAEDLAIYYCQQYYDTPVTFGSGTKLELK

SEQ ID NO: 145 - protein, 112 aa, Artificial sequence
  synthetic sequence
AIQVTQSPNSLSASLGDRVTLTCRASQDINNKMAWYQQKPGEVPQLLIYSASTLQSGTPSRFSGSGSGTDFSFTISHLQSEDFATYYCLQGYSLYTFGAGTKLELKRADAAP

SEQ ID NO: 146 - protein, 107 aa, Artificial sequence
  synthetic sequence
DIQMTQSPSNLAASPGESVSINCKASKSISKFLAWYQQKSGKANKLLIYSASTLQSGTPSRFSGSGSGTDFTLTIRNLEPEDFGLYYCQQHNEYPLTFGSGTKLEIK

SEQ ID NO: 147 - protein, 112 aa, Artificial sequence
  synthetic sequence
DVVMTQTPPSLSVAIGQSVSISCKSSQSLVYSDGKTYLHWLLENSGRSPKRLIYRVSNLDSGVPDRFSGTGSQKDFTLKISRVEAQDLGVYYCAQTTHFPWTFGGGTKLELK

SEQ ID NO: 148 - protein, 107 aa, Artificial sequence
  synthetic sequence
DVVMTQSPSHLAASPGESVSINCKASKNIYKYLAWYQQKPGKTNKLLIYSGSTLQSGTPSRFSGSGSGSDFTLTIRNLEPEDFGLYYCQHHNEYPPTFGGGTKLEIK

SEQ ID NO: 149 - protein, 106 aa, Artificial sequence
  synthetic sequence
DIVLTQSPTTMAAFPGEKVTITCRASSSVSYMYWYQQKSGASPKSWIYETSKLASGVPDRFSGSGSGTSYSFTISSMETEDAATYYCHQWSSIPLTFGSGTKLEIK

SEQ ID NO: 150 - protein, 107 aa, Artificial sequence
  synthetic sequence
DVVMTQSPSNLAASPGESVSINCKASKSIGKFLAWYQQKPGRPNKLLIYSGSTLQSGTPSRFSGSGSGTDFTLTIRNLEPGDFGLYYCQHHIEYPPTFGPGTKLELK

SEQ ID NO: 151 - protein, 107 aa, Artificial sequence
  synthetic sequence
DIWMTQSPTSMSISVGDRVTMNCKASQNVGSNVDWYQQKTGQSPKLLIYKASNRYTGVPDRFTGSGSGTDFTFTISNMQAEDLAVYYCMQSNSYPWTFGGGTKLELK

SEQ ID NO: 152 - protein, 107 aa, Artificial sequence
  synthetic sequence
DIWMTKSPSSMSASLGDRVTITCQASQDIGNNLIWFQQKPGKSPRRMIYYATNLANGVPSRFSGSRSGSDYSLTIISLESEDMADYHCLQYKQYPYTFGAGTKLEIK

SEQ ID NO: 153 - protein, 107 aa, Artificial sequence
  synthetic sequence
DIQMTQSPSNLAASPGESVSINCKASETIYKYLAWYQQKPGRTNKLLIYSGSTLQSGTPSRFSGSGSGTDFTLTIRNLEPEDFGRYYCQHHVEYPPTFGAGTKLELK

SEQ ID NO: 154 - protein, 106 aa, Artificial sequence
  synthetic sequence
TPPSLSVAIGQSVSISCKSSQSLVYSDGKTYLHWLLQISGRSPERLIYQVSNLDSGVPDRFSGTGSQKDFTLKISRVEAKDLGFYYCAQTTHFPYTFGAGTKLELK

SEQ ID NO: 155 - protein, 16 aa, Artificial sequence
  synthetic sequence
RSSQSLLDSDGTTYLY

SEQ ID NO: 156 - protein, 16 aa, Artificial sequence
  synthetic sequence
RSSQSLVHSNGNTYLH

SEQ ID NO: 157 - protein, 11 aa, Artificial sequence
  synthetic sequence
LTSEDISSYLT

SEQ ID NO: 158 - protein, 11 aa, Artificial sequence
  synthetic sequence
RASQDINNKMA

SEQ ID NO: 159 - protein, 16 aa, Artificial sequence
  synthetic sequence
KSSQSLVYSDGKTYLH

SEQ ID NO: 160 - protein, 11 aa, Artificial sequence
  synthetic sequence
RASQDIGNYLT

SEQ ID NO: 161 - protein, 17 aa, Artificial sequence
  synthetic sequence
KSSQSLLYSGNQKNSLA

SEQ ID NO: 162 - protein, 11 aa, Artificial sequence
  synthetic sequence
RASENINNYLS

SEQ ID NO: 163 - protein, 15 aa, Artificial sequence
  synthetic sequence
KTNQNVDYYGNSYIH

SEQ ID NO: 164 - protein, 16 aa, Artificial sequence
  synthetic sequence
RSSQSLLDSDGNTYLY

SEQ ID NO: 165 - protein, 11 aa, Artificial sequence
  synthetic sequence
RASESVTSFMN

SEQ ID NO: 166 - protein, 11 aa, Artificial sequence
  synthetic sequence
KASKSISKYLA

SEQ ID NO: 167 - protein, 11 aa, Artificial sequence
  synthetic sequence
KGSQNINNFLA

SEQ ID NO: 168 - protein, 10 aa, Artificial sequence
  synthetic sequence
RASFRVSYMH

SEQ ID NO: 169 - protein, 11 aa, Artificial sequence
  synthetic sequence
QASQNINKYIA

SEQ ID NO: 170 - protein, 16 aa, Artificial sequence
  synthetic sequence
QSSKSLLYSDGKTYLN

SEQ ID NO: 171 - protein, 17 aa, Artificial sequence
  synthetic sequence
RSSQSLLYSGNQKNYLA

SEQ ID NO: 172 - protein, 17 aa, Artificial sequence
  synthetic sequence
KSSQSLLYSGNQKNYLA

SEQ ID NO: 173 - protein, 15 aa, Artificial sequence
  synthetic sequence
RARQSVSISRYNLMH

SEQ ID NO: 174 - protein, 15 aa, Artificial sequence
  synthetic sequence
WASQSVSISRYNLMH

SEQ ID NO: 175 - protein, 11 aa, Artificial sequence
  synthetic sequence
KASKSISKFLA

SEQ ID NO: 176 - protein, 11 aa, Artificial sequence
  synthetic sequence
KASKNIYKYLA

SEQ ID NO: 177 - protein, 10 aa, Artificial sequence
  synthetic sequence
RASSSVSYMY

SEQ ID NO: 178 - protein, 11 aa, Artificial sequence
  synthetic sequence
KASKSIGKFLA

SEQ ID NO: 179 - protein, 11 aa, Artificial sequence
  synthetic sequence
KASQNVGSNVD

SEQ ID NO: 180 - protein, 11 aa, Artificial sequence
  synthetic sequence
QASQDIGNNLI

SEQ ID NO: 181 - protein, 11 aa, Artificial sequence
  synthetic sequence
KASETIYKYLA

SEQ ID NO: 182 - protein, 7 aa, Artificial sequence
  synthetic sequence
LVSNLGS

SEQ ID NO: 183 - protein, 7 aa, Artificial sequence
  synthetic sequence
RISNRFS

SEQ ID NO: 184 - protein, 7 aa, Artificial sequence
  synthetic sequence
GANSLQA

SEQ ID NO: 185 - protein, 7 aa, Artificial sequence
  synthetic sequence
YASTLQS

SEQ ID NO: 186 - protein, 7 aa, Artificial sequence
  synthetic sequence
QVSNLDS

SEQ ID NO: 187 - protein, 7 aa, Artificial sequence
  synthetic sequence
GATNLAA

SEQ ID NO: 188 - protein, 7 aa, Artificial sequence
  synthetic sequence
WASTRQS

SEQ ID NO: 189 - protein, 7 aa, Artificial sequence
  synthetic sequence
HTSRLQP

SEQ ID NO: 190 - protein, 7 aa, Artificial sequence
  synthetic sequence
LASNLAS

SEQ ID NO: 191 - protein, 7 aa, Artificial sequence
  synthetic sequence
LASDLES

SEQ ID NO: 192 - protein, 7 aa, Artificial sequence
  synthetic sequence
SGSTLQS

SEQ ID NO: 193 - protein, 7 aa, Artificial sequence
  synthetic sequence
KTNSLHT

SEQ ID NO: 194 - protein, 7 aa, Artificial sequence
  synthetic sequence
DTSKLAS

SEQ ID NO: 195 - protein, 7 aa, Artificial sequence
  synthetic sequence
YTSTLES

SEQ ID NO: 196 - protein, 7 aa, Artificial sequence
  synthetic sequence
WMSIRAS

SEQ ID NO: 197 - protein, 7 aa, Artificial sequence
  synthetic sequence
WSSTRQY

SEQ ID NO: 198 - protein, 7 aa, Artificial sequence
  synthetic sequence
WSSTRQS

SEQ ID NO: 199 - protein, 7 aa, Artificial sequence
  synthetic sequence
RASNLAS

SEQ ID NO: 200 - protein, 7 aa, Artificial sequence
  synthetic sequence
SASTLQS

SEQ ID NO: 201 - protein, 7 aa, Artificial sequence
  synthetic sequence
RVSNLDS

SEQ ID NO: 202 - protein, 7 aa, Artificial sequence
  synthetic sequence
ETSKLAS

SEQ ID NO: 203 - protein, 7 aa, Artificial sequence
  synthetic sequence
KASNRYT

SEQ ID NO: 204 - protein, 7 aa, Artificial sequence
  synthetic sequence
YATNLAN

SEQ ID NO: 205 - protein, 9 aa, Artificial sequence
  synthetic sequence
MQATHVPFT

SEQ ID NO: 206 - protein, 9 aa, Artificial sequence
  synthetic sequence
FQSTHFPHT

SEQ ID NO: 207 - protein, 9 aa, Artificial sequence
  synthetic sequence
QQSYKFPLT

SEQ ID NO: 208 - protein, 8 aa, Artificial sequence
  synthetic sequence
LQGYSLYT

SEQ ID NO: 209 - protein, 9 aa, Artificial sequence
  synthetic sequence
AQTTHFPFT

SEQ ID NO: 210 - protein, 9 aa, Artificial sequence
  synthetic sequence
LQSIQYPFT

SEQ ID NO: 211 - protein, 9 aa, Artificial sequence
  synthetic sequence
QHYFDTPYT

SEQ ID NO: 212 - protein, 8 aa, Artificial sequence
  synthetic sequence
QQGYTPPT

SEQ ID NO: 213 - protein, 9 aa, Artificial sequence
  synthetic sequence
QQSRNLPFT

SEQ ID NO: 214 - protein, 8 aa, Artificial sequence
  synthetic sequence
MQATHSYT

SEQ ID NO: 215 - protein, 9 aa, Artificial sequence
  synthetic sequence
QQSWIDPPT

SEQ ID NO: 216 - protein, 9 aa, Artificial sequence
  synthetic sequence
QHHNEYPPT

SEQ ID NO: 217 - protein, 8 aa, Artificial sequence
  synthetic sequence
YQYNNGYT

SEQ ID NO: 218 - protein, 9 aa, Artificial sequence
  synthetic sequence
QQWTSNSRT

SEQ ID NO: 219 - protein, 8 aa, Artificial sequence
  synthetic sequence
LQYVNLYT

SEQ ID NO: 220 - protein, 9 aa, Artificial sequence
  synthetic sequence
QQFLEFPYT

SEQ ID NO: 221 - protein, 9 aa, Artificial sequence
  synthetic sequence
QQYYDTPLT

SEQ ID NO: 222 - protein, 9 aa, Artificial sequence
  synthetic sequence
QQSRDSPWT

SEQ ID NO: 223 - protein, 9 aa, Artificial sequence
  synthetic sequence
QQSGESPWT

SEQ ID NO: 224 - protein, 9 aa, Artificial sequence
  synthetic sequence
QQYYDTPVT

SEQ ID NO: 225 - protein, 9 aa, Artificial sequence
  synthetic sequence
QQHNEYPLT

SEQ ID NO: 226 - protein, 9 aa, Artificial sequence
  synthetic sequence
AQTTHFPWT

SEQ ID NO: 227 - protein, 9 aa, Artificial sequence
  synthetic sequence
HQWSSIPLT

SEQ ID NO: 228 - protein, 9 aa, Artificial sequence
  synthetic sequence
QHHIEYPPT

SEQ ID NO: 229 - protein, 9 aa, Artificial sequence
  synthetic sequence
MQSNSYPWT

SEQ ID NO: 230 - protein, 9 aa, Artificial sequence
  synthetic sequence
LQYKQYPYT

SEQ ID NO: 231 - protein, 9 aa, Artificial sequence
  synthetic sequence
QHHVEYPPT

SEQ ID NO: 232 - protein, 9 aa, Artificial sequence
  synthetic sequence
AQTTHFPYT

SEQ ID NO: 233 - protein, 1325 aa, Homo sapiens
MLPVYQEVKPNPLQDANLCSRVFFWWLNPLFKIGHKRRLEEDDMYSVLPEDRSQHLGEELQGFWDKEVLRAENDAQKPSLTRAIIKCYWKSYLVLGIFTLIEESAKVIQPIFLGKIINYFENYDPMDSVALNTAYAYATVLTFCTLILAILHHLYFYHVQCAGMRLRVAMCHMIYRKALRLSNMAMGKTTTGQIVNLLSNDVNKFDQVTVFLHFLWAGPLQAIAVTALLWMEIGISCLAGMAVLIILLPLQSCFGKLFSSLRSKTATFTDARIRTMNEVITGIRIIKMYAWEKSFSNLITNLRKKEISKILRSSCLRGMNLASFFSASKIIVFVTFTTYVLLGSVITASRVFVAVTLYGAVRLTVTLFFPSAIERVSEAIVSIRRIQTFLLLDEISQRNRQLPSDGKKMVHVQDFTAFWDKASETPTLQGLSFTVRPGELLAVVGPVGAGKSSLLSAVLGELAPSHGLVSVHGRIAYVSQQPWVFSGTLRSNILFGKKYEKERYEKVIKACALKKDLQLLEDGDLTVIGDRGTTLSGGQKARVNLARAVYQDADIYLLDDPLSAVDAEVSRHLFELCICQILHEKITILVTHQLQYLKAASQILILKDGKMVQKGTYTEFLKSGIDFGSLLKKDNEESEQPPVPGTPTLRNRTFSESSVWSQQSSRPSLKDGALESQDTENVPVTLSEENRSEGKVGFQAYKNYFRAGAHWIVFIFLILLNTAAQVAYVLQDWWLSYWANKQSMLNVTVNGGGNVTEKLDLNWYLGIYSGLTVATVLFGIARSLLVFYVLVNSSQTLHNKMFESILKAPVLFFDRNPIGRILNRFSKDIGHLDDLLPLTFLDFIQTLLQVVGVVSVAVAVIPWIAIPLVPLGIIFIFLRRYFLETSRDVKRLESTTRSPVFSHLSSSLQGLWTIRAYKAEERCQELFDAHQDLHSEAWFLFLTTSRWFAVRLDAICAMFVIIVAFGSLILAKTLDAGQVGLALSYALTLMGMFQWCVRQSAEVENMMISVERVIEYTDLEKEAPWEYQKRPPPAWPHEGVIIFDNVNFMYSPGGPLVLKHLTALIKSQEKVGIVGRTGAGKSSLISALFRLSEPEGKIWIDKILTTEIGLHDLRKKMSIIPQEPVLFTGTMRKNLDPFNEHTDEELWNALQEVQLKETIEDLPGKMDTELAESGSNFSVGQRQLVCLARAILRKNQILIIDEATANVDPRTDELIQKKIREKFAHCTVLTIAHRLNTIIDSDKIMVLDSGRLKEYDEPYVLLQNKESLFYKMVQQLGKAEAAALTETAKQVYFKRNYPHIGHTDHMVTNTSNGQPSTLTIFETAL

SEQ ID NO: 234 - protein, 5 aa, Artificial sequence
  synthetic sequence
GSGGS

SEQ ID NO: 235 - protein, 4 aa, Artificial sequence
  synthetic sequence
GGGS

SEQ ID NO: 236 - protein, 4 aa, Artificial sequence
  synthetic sequence
GGSG

SEQ ID NO: 237 - protein, 5 aa, Artificial sequence
  synthetic sequence
GGSGG

SEQ ID NO: 238 - protein, 5 aa, Artificial sequence
  synthetic sequence
GSGSG

SEQ ID NO: 239 - protein, 5 aa, Artificial sequence
  synthetic sequence
GSGGG

SEQ ID NO: 240 - protein, 5 aa, Artificial sequence
  synthetic sequence
GGGSG

SEQ ID NO: 241 - protein, 5 aa, Artificial sequence
  synthetic sequence
GSSSG